(19) United States
(12) Patent Application Publication
(10) Pub. No.: US 2024/0226393 A9
(43) Pub. Date: Jul. 11, 2024

(54) AUXETIC WEB STRUCTURE OR FIELD STRUCTURE, AND USE

(71) Applicant: Christian-Albrechts-Universitaet zu Kiel, Kiel (DE)

(72) Inventors: Sabrina M. Curtis, Frederick, MD (US); Duygu Dengiz, Reinbek (DE); Eckhardt Quandt, Heikendorf (DE)

(21) Appl. No.: 18/277,636

(22) PCT Filed: Jan. 7, 2022
(86) PCT No.: PCT/DE2022/100001

§ 371 Date: Aug. 17, 2023

(30) Foreign Application Priority Data
Feb. 20, 2021 (DE) 10 2021 104 057.4
Feb. 20, 2021 (DE) 10 2021 104 058.2

(51) Int. Cl. A61L 31/14; B29C 44/34

(52) U.S. Cl. CPC A61L 31/14; B29C 44/357

(57) ABSTRACT

An auxetic web structure or field structure useful for a medical implant, combining the advantageous mechanical properties of auxetic structures and stretchable interconnects in such a way as to provide an auxetic structure which is particularly stretchable, while at the same time providing sufficiently large areas which are subjected at most to only minor stresses. This structure takes advantage of the superior mechanical properties offered by auxetic metamechanical materials (hereafter referred to as auxetic) and improves their stretchability and compressibility by incorporating interconnects into the auxetic framework.

The invention relates to an auxetic web structure or field structure comprising island structure areas and connections between the individual island structure areas, wherein open spaces are present between the individual island structure areas and the connections with the island structure areas form a web structure or field structure and the connections are designed to be stretchable.

Such structures can also be referred to as auxetic metamechanical materials with improved extensibility and compressibility.

Three recent review papers, namely:
Prawoto, Yunan. “Seeing auxetic materials from the mechanics point of view: a structural review on the negative Poisson's ratio.” Computational Materials Science 58 (2012): 140-153;
Ren, Xin, et al. “Auxetic metamaterials and structures: a review.” Smart materials and structures 27.2 (2018): 023001.
Saxena, Krishna Kumar, Raj Das, and Emilio P. Calius. “Three decades of auxetics research-. materials with negative Poisson's ratio: a review.” Advanced Engineering Materials 18.11 (2016): 1847-1870 and
Wang, Zhenwei, et al. “Progress in Auxetic Mechanical Metamaterials: Structures, Characteristics, Manufacturing Methods, and Applications.” Advanced Engineering Materials 22.10 (2020): 2000312
   summarize the extensive advances in auxetic structures over the past three decades from over 300 research papers related to modeling, fabrication, and characterization of auxetic structures.

The use of stretchable compounds to improve the mechanical strain properties of auxetic structures is not mentioned in these reviews.

In “Non-planar shearing auxetic structures, devices, and methods,” US 2018/0311833A1 describes auxetic structures as “A non-planar shearing auxetic structure, comprising each unit cell is defined by a plurality of elements and by an internal angle between two of the plurality of elements that are connected together by a pivot joint,” where the pivot joint disclosed and described there is, in principle, the site of the modified extensible joint also disclosed herein.

Hassanin, Hany et al. disclose in “4D printing of NiTi auxetic structure with improved ballistic performance” Micromachines 11.8 (2020): 745 the development of a NiTi structure with a negative Poisson ratio with superelasticity/shape memory for improved ballistic applications. A 4D printing process is used to fabricate the optimized auxetic NiTi structure.

The only auxetic design that appears to be similar to interconnect geometries used in stretchable electronics today is the rotachiral design. It resembles the “2D wavy network constructed with horseshoe building blocks, configured into a triangle lattice geometry” proposed by K-I Jang et al. in “Soft network composite materials with deterministic and bio-inspired designs” Nature Communications 6, 6566 (2015). The authors note that a negative effect of Poisson's ratio of the structure is observed at low strains, but the auxetic behavior of the stretchable mesh with triangular lattice geometry is not investigated. No negative Poisson's ratio is observed for the square lattice geometry in this paper. Moreover, the proposed wavy mesh designs do not provide a large island space available for possible functionalization electronic components.

2D thin film electronic devices that are intended to serve as contact points to 3D biological complex surfaces must be able to withstand large macroscopic stresses without compromising the functional components of the device. In the field of flexible and stretchable electronics, numerous solutions to this problem have been found by reconfiguring the geometry of the entire electronic circuit. One solution is to turn the electronic circuit into an undulating 2D thin-film metal mesh. Another popular technique is the “island bridge,” which structures a series of periodic islands connected in the middle by stretchable links. The stresses on the islands remain low even when deformed because the stresses and strains are concentrated on the extensible connections. Examples of such connections known from prior art include serpentines, fractal patterns, 2D wave lattices, and more recently Archimedean spiral structures connected at the center of islands. State-of-the-art “island-bridge” structures are designed to expand under biaxial loading. These solutions allow for many impressive devices. However, there are some limitations to traditional stretchable designs, including a positive (or zero) Poisson's ratio. In addition, it is usually necessary for traditional stretchable electronics to be integrated on an elastomeric substrate to promote elastic recovery. There is a need to configure 2D electronic devices into free-standing structural geometries that allow the device to elastically achieve large macroscopic strains, conformally integrate into 3D surfaces, and maintain high areal density for functional electronic components.

The U.S. Pat. No. 8,552,299 B2, electronic circuits based on the island-bridge layout, which consists of an array of periodic islands connected by serpentines, are shown. The structures shown have no auxetic properties and also have a Poisson number of zero or positive. In some cases, the fabricated devices mimic mechanical behavior similar to auxetic structures (i.e., ability to bend over a dome-shaped curvature, extension under biaxial (x and y direction) loading). In most cases, however, the serpentine junctions bend out-of-plane at equilibrium, meaning that the junctions are not in conformal contact with the underlying substrate during bending deformation. High-performance bioelectrodes (i.e., skin sensors) require full conformal contact (i.e., perfect interface) with the skin to maximize signal acquisition.

U.S. Pat. No. 10,192,830 B2 also suggests useful 2D designs of self-similar and fractal designs to develop stretchable interconnects based on serpentine structures. These fractal interconnects have been shown to improve the maximum strain that circuits can achieve when arranged in an island-bridge layout. The use of in-plane fractal designs likely improves the out-of-plane conformance problem created by kinked serpentine structures. The use of fractal designs for auxetic structures is not mentioned. In addition, 2D crosslinking materials with a zero or positive Poisson's ratio are demonstrated that are based on the serpentine shape and arranged in a fractal pattern. It is not mentioned that these mesh materials exhibit auxetic behavior, moreover, there are no periodic large-area islands as part of the structure for device integration.

The mechanical properties of auxetic materials make them ideal platforms for stretchable electronics to be integrated into the body. The problem with conventional auxetic structures is that the maximum strain that can be achieved is relatively low and is limited by the stiffness of the interconnect. In Grima, Joseph N., et al. “Auxetic behavior from stretching connected squares.” Journal of Materials Science 43.17 (2008): 5962-5971, strain behavior in rotating square models is studied. The squares are connected at the corners by hinged joints which, if assumed to be perfectly rigid, can easily rotate relative to each other to exhibit auxetic behavior under applied deformation. These squares can also produce auxetic behavior by lengthening (or shortening) the sides of the squares through the stretching mechanism. In Grima, Joseph N., Elaine Manicaro, and Daphne Attard. “Auxetic behavior of connected squares and rectangles of different sizes.” Proceedings of the Royal Society A: Mathematical, Physical and Engineering Sciences 467.2126 (2011): 439-458, it is shown that rotating squares and rectangles of different sizes can be connected and still maintain auxetic behavior.

Some examples of traditional auxetic structures that could be modified to have improved ductility/compressibility include honeycombs, chiral (circle, square, hexagon, etc.), triangular structures and star, diamond lattice (square and oblong), origami-based metamaterials, kirigami-based metamaterials, key-bricked structures, slot-perforated structures, rotating rigid structures, rotating semi-rigid structures, and hierarchical auxetic systems.

U.S. Pat. No. 8,883,287 B2 discloses structures that resemble “rotating squares” auxetic structures. In this printing, the rigid connection points between the squares are replaced by connection points with a strain relief function, which have the same shape as a serpentine connection, similar to the “island bridge structures” known from stretchable electronics. However, the structures presented here are not called auxetic or assigned a negative Poisson's ratio. Furthermore, the structures must be stretched in both the x- and y-directions (biaxially), rather than uniaxially, to achieve expansion. In other words, this means that the structures presented in the paper are not auxetic. The “island interconnect structures” known from classical stretchable electronics can also only expand under biaxial stretching.

From the prior art, auxetic framework structures are known as flexible substrates for functional components, in particular for electronic components such as solar cells, antennas, batteries and sensors. In this regard, reference is made, for example, to US 2010/330 338 A1, US 2018/061 743 A1 and US 2020/144 431 A1. The aforementioned publications disclose auxetic substrates made of classical substrate materials such as the semiconductor silicon or a polymer plastic, which themselves do not exhibit any significant electrical conductivity.

Furthermore, it is sufficiently known in the prior art to manufacture implants, in particular stents, from SMA based on NiTi alloys.

It is further widely known in the prior art to functionalize the surfaces of such medical implants with coatings.

Conventional auxetic structures can be modified in various ways to meet the requirements of a medical implant. Large-area islands can be periodically integrated into the framework of conventional auxetic geometries known to the prior art. In addition, the serrated hinges of large-area auxetic geometries (e.g., rotating polygons) can be modified with more compliant structures (e.g., serpentines) to allow for larger deformations.

Xue, et al. address the relationship between stent structures and clinical outcomes in the treatment of coronary artery disease in “Design of self-expanding auxetic stents using topology optimization” Frontiers in Bioengineering and Biotechnology 8 (2020): 736. First, a method for simultaneous topological optimization is developed to systematically find the best material distribution within the design domain. An extended parametric level set method with shell elements is proposed for topology optimization to ensure computational accuracy and efficiency. Then, an auxetic metamaterial with negative Poisson's ratio is introduced in self-expanding stents. Auxetics can improve the mechanical properties of the structure, such as fracture toughness, identification and shear strength, and vibration energy absorption, which helps to address the drawbacks due to mechanical failures. Finally, the optimized self-expanding stent is numerically validated using ANSYS commercial software and then prototyped using additive manufacturing techniques.

US 2017/0 007 400 A1 discloses a composite biomaterial comprising a continuous metal sheet with curved elements defining a first window pattern, and a polymer layer over at least one surface of the continuous metal sheet. The bent elements are elastically stretchable so that the continuous metal sheet can bend in more than one axis without buckling or folding.

Modern stretchable microelectronic mechanical systems (MEMS) are typically based on wave-meshed thin-film metals and island bridge layouts with thin-film serpentine interconnects. Thanks to the serpentine and wavy structures, extremely large uniaxial global strains can be achieved. The major drawback of modern stretchable electronics is that the stretchable structures have a positive or zero Poisson's ratio, which means that the structure either contracts or does not change in the perpendicular direction under uniaxial tensile loading.

Next-generation electronic devices to be integrated into biological tissue must be minimally invasive, ultra-conformal, and allow for co-integration of complex microelectronic circuitry. Electronics to be integrated on various body surfaces, e.g., blood veins, blood vessels, skin, heart valves, etc., must be able to withstand the extreme stretching, bending, and twisting deformations associated with the body's natural curvature changes. For example, electronic tattoos worn directly on the skin, i.e., skin sensors, and medical implants inserted inside the body should ideally conform to their environment to optimize device performance. It is also desirable for the structure to have large low-stress regions to accommodate and electrically connect various MEMS components. An additional requirement for most medical implants, e.g., stents, is that they must also be able to compress and expand by at least ˜4 times their volume to be squeezed to a small enough diameter of microcatheter to be deployed by noninvasive endovascular treatments.

The major disadvantage of the state of the art in auxetic structures is that the extensibility and compressibility of the entire structure is limited by the stiffness of the connecting hinges (or pivot joints) of the structures. The stiffness of traditional auxetic structures may limit their practical use as implantable medical devices or other stretchable electronic applications because volume expansion/compression requirements cannot be met with traditional designs.

Further, in the prior art, electronic components have already been connected to SMA films, and stents with auxetic scaffolds have been proposed, which can also be formed from an SMA material in an obvious manner.

The problem with the devices known in the prior art is that the one required implantable structure with electronic components in the form described hereafter is not known, but such a structure is needed.

The main challenge in functionalizing conventional stent designs (that is, lattice structures, traditional auxetic structures, synthetic composite structures) is the small area available for integrating electrical devices. The next leap in quality of medical care requires long-term, non-invasive, unobtrusive, continuous monitoring of vital biomedical signs. Electronic sensors that can be integrated on various surfaces of the body, e.g., blood veins/vessels/arteries, heart valves, skin, bones, etc., must adapt to these complex surfaces and withstand the extreme stretching, bending and twisting deformations associated with the body's natural curvature changes. The main problem with conventional electronic components is that they are rigid and typically cannot adapt to curved surfaces.

The next generation of medical implants will be functionalized to wirelessly transmit patient data to physicians in real time, enabling better quality of treatment. Conventional stents based on woven, braided and laser-cut shape memory alloy stents are somewhat rigid and cannot fully conform to the shape of the vein. In addition, conventional stents do not provide as high an area fraction of stress-free regions as could accommodate active or passive functional components. This feature is necessary for future miniaturized functionalized medical implants to allow integration for wireless transmission antennas, energy harvesting devices, energy storage devices, and other types of sensor or actuator components.

U.S. Pat. No. 10,660,200 B2 discloses that Archimedean spiral designs are significantly more stretchable than serpentine and fractal designs currently used for stretchable electronics. Archimedean spirals exhibit significantly lower stresses than serpentine interconnects. They can thus achieve large uniaxial displacements by buckling and twisting out of plane. The structures disclosed in the print connect the spirals through a well-known island-bridge layout, where the Archimedean spiral connects the island directly in the center of the surface. The connection point at the center of the face of the islands is the reason that the structure as a whole has a positive or zero Poisson's ratio.

The problem of lower extensibility of auxetic structures described above can be solved by modifying the rigid hinge or pivot joint of an auxetic structure with extensible joints. A first example of this can be found in US 2020/0144431 A1, which shows that the mechanical properties of auxetic structures can be improved by using serpentine joints. The publication discloses a deformable array of semiconductor devices and a method of fabricating such a deformable array. The deformable array includes a plurality of islands, each island including at least one semiconductor device, and the plurality of islands are arranged in an auxetic geometry. The interconnections of the islands with each other are formed as serpentines, as mentioned above, and each serpentine also includes a semiconductor device. The structure disclosed herein is capable of expanding under uniaxial loading.

The object of the present invention is to combine the advantageous mechanical properties of auxetic structures and stretchable interconnects in such a way as to provide an auxetic structure which is particularly stretchable, while at the same time providing sufficiently large areas which are subjected at most to only minor stresses.

The present invention solves the above technical problems by presenting a mechanically stretchable auxetic structure. This structure takes advantage of the superior mechanical properties offered by auxetic metamechanical materials (hereafter referred to as auxetic) and improves their stretchability and compressibility by incorporating interconnects into the auxetic framework.

Auxetic materials are known periodic structures characterized by a negative Poisson's ratio, meaning they expand laterally when stretched longitudinally. Under a uniaxial tensile force, these structures expand or contract in the perpendicular direction of the applied force. The structures exhibit impressive material properties, including synclastic bending, high stiffness-to-weight ratio, large shear strength, large hardness values, high indentation/impact strength, reduced fatigue crack propagation, greater toughness and modulus resistance, and vibration damping. In particular, synclastic bending offers unique opportunities for wearable electronics and medical device applications by enabling conformal bending around concave, convex, as well as convex bending around concave, convex, dome-shaped curves and other complex, nonlinear 3D geometries, allowing two-dimensional auxetic structures to conform to any three-dimensional surface.

In particular, expansion/compression requirements should be met by the auxetic structure presented here, which are needed, for example, by functionalized medical implants. Thus, a flexible, very often bendable substrate for electronic components shall be shown, which has the property to be biocompatible and implantable, so that body movements are allowed and the substrate and its structure as well as the electronic components located thereon are not destroyed or damaged by movements.

Another task can be seen in complementarily realizing or using the combination of shape memory alloys as the backbone of the auxetic structure for a new design for medical devices that can be used in the heart and brain through non-invasive endovascular treatments.

These tasks are solved with an auxetic web structure or field structure according to the main claim.

The auxetic web structure or field structure has island structure areas and connections between the individual island structure areas, wherein open spaces are present between the individual island structure areas and the connections form a web or field structure with the island structure areas, wherein that the connections are stretchable and are designed as Archimedean spiral connections and/or self-similar fractal design connections, the connections intersect the island structure areas at the same location and at the same angle as the previous rigid connectors intersect the auxetic framework, the island structure areas are not or only slightly bendable under the action of force, the stresses on the island structure areas being at least one order of magnitude below those of the extensible connection, and/or are not or only insignificantly variable in magnitude, and the Poisson's ratio of the structure under uniaxial deformation is negative.

Thus, extensible structural elements are provided by combining auxetic geometries from the literature with extensible joints. The auxetic structures are characterized by a negative Poisson's ratio, which means that the structure expands under uniaxial tensile loading in the perpendicular direction of the applied force.

Similarly, the structures presented here also contract in the perpendicular direction under uniaxial compressive loading due to the negative Poisson's ratio.

Any thin film material incorporated into the structures disclosed in the present invention exhibits auxetic properties with improved extensibility and compressibility.

The subject matter of the invention allows for even higher extensibility than disclosed in US 2020/0144431 A1, as the serpentine joints described in said disclosure are replaced by joints known to exhibit higher extensibility, namely self-similar and/or fractal design joints and/or Archimedean spiral joints. Theoretically, the mechanical properties of many other auxetic structures could be improved by replacing their rigid connections with stretchable ones.

The Archimedean spiral structure has the largest elastic extensibility of up to 200%, while the regular serpentine and self-similar serpentine have elastic extensibilities of 112% and 98%, respectively. Therefore, extensible auxetic structures with Archimedean joints can achieve tremendous extensibility and larger volume and area expansions compared to auxetic structures with extensible serpentine joints, fractal joints, and conventional rigid hinges. Moreover, the desired properties (such as extensibility, etc.) of the auxetic web structure or field structure according to the invention are further improved by the fact that the joints intersect the island structure areas at the same location and angle as the previous rigid connectors intersect the auxetic framework.

The present invention achieves greater radial expansion and smaller radial compression compared to conventional auxetic structures. This is achieved by replacing the rigid connection points of the island structure areas with flexible/stretchable Archimedean spiral connections or self-similar fractal design connections. Stretchable auxetic structures could be formed by different sized island structure areas in combination with different sized/shaped stretchable intermediate connections.

Any material processed into this type of stretchable auxetic structure can benefit from improved material properties. Due to the negative Poisson's ratio, auxetic structures can conform to almost any concave or convex surface. This property results in synclastic bending, which allows auxetic structures to conform to nonlinear surfaces. By replacing the traditional “swivels” or rigid connection points of traditional auxetic structures with sinuous web connections, namely in particular self-similar fractal design connections and/or Archimedean spirals, the ductility and compressibility of the auxetic structure is improved by several orders of magnitude.

In this context, the connections can additionally exhibit the following properties, at least in sections: arc-shaped and/or kink-free and/or with bending radii and/or bifilar and/or helical.

Archimedean-inspired designs for ductile joints are very advantageous, as it has already been shown that they can achieve ductility greater than 1020% if the joint is designed with a high amplitude/width ratio. It is believed that a uniform and small curvature contributes to a greater ductility of such structures. In addition, complex spiral shapes that include at least parts of known spirals would also be conceivable.

In particular, the individual island structure areas and/or the individual connections of the auxetic web structure or field structure can be designed to vary in size and shape. Thus, an overall design of the auxetic structure adapted to the requirements becomes possible.

The auxetic web structure or field structure can additionally be formed identically and/or periodically at least in sections over areas.

The island structure areas can additionally be formed in the same way. The island structure areas can occupy a large area, whereby stress-free expansion is still possible due to the connections. The island structure areas can be sized to accommodate any type of conventional rigid electronic MEMS component. The stretchable auxetic structure can be designed to compromise between the density of the “unit cell area” and the maximum volume expansion of the entire structure.

In particular, the self-similar fractal design connections may include Koch's lines, Peano's lines, Hilbert's lines, Moore's loops, Vicsek's loops, and branched meshes such as the Greek cross.

The auxetic web structure or field structure can be made planar, 2D, and subsequently adapted to 3D surfaces while maintaining a 3D auxetic behavior. This is particularly possible if the 2D fabricated structure is formed from an amorphous shape memory alloy.

The open spaces between the island structure areas and the interconnects can be mesh-like and potentially serve as a space for further functionalization of the device or simply as a stretchable interconnect.

Preferably, the auxetic web structure or field structure may include at least individual island structure areas for receiving electronics or individual island structure areas populated with electronics.

The electronics located on the island structure areas may be electrically connected to each other via at least one conductive path electrically insulated from the auxetic web structure or field structure on at least one connection of the auxetic web structure or field structure.

In particular, the auxetic web structure or field structure according to the invention may be used as an implantable structure made of a biocompatible material, wherein the biocompatible material is a metallic shape memory alloy (SMA) and the auxetic web structure or field structure is metallic.

Here, the implantable structure may have a self-expanding implant shape with a cylindrical and/or spherical and/or hemispherical and/or tubular and/or curved tubular structure at least in sections.

The auxetic web structure and field structure according to the invention thus provide an extremely deformable metallic auxetic substrate with large areas, which is suitable for the integration of functional electronic devices.

In this context, the electronic devices can also be brittle, e.g. comprising ceramics, and nevertheless remain intact and adhered to the island structure areas during framework bending.

Metal connections with good electrical conductivity between different islands or island structure areas of a scaffold structure must be precisely applied to the connections between the island structure areas, which becomes increasingly costly and complex with smaller structure dimensions. Generally, electronic components or devices are at least two-terminal devices that generate electrical voltages or respond to voltage signals. It is therefore desirable, and practically unavoidable when a large part of the island structure areas are occupied by components, that each interconnection of a framework structure carries at least two metallic but mutually electrically isolated paths for signal conduction. The insulation of the two paths must not be too thin, since even low voltages can otherwise lead to locally high field strengths and thus electrical breakdowns. Particularly in the case of scaffold structures intended for implantation in a patient's vessels, the structure dimensions must be small and the widths of the connections/bridges a fortiori must be designed in the range of less than ten micrometers. When narrow webs are occupied by more than one metallic conductor, the voltage signals that can be transmitted through the network are severely limited. For safety reasons, this can lead to restrictions in the selection of electronic components and/or in the design of the network architecture.

By fabricating circuits on auxetically structured shape memory alloy substrates, extreme flexibility is enabled within the overall electronic system. The advantageous material properties of shape memory alloy (SMA) coupled with the superior mechanical properties of auxetic structures allow the substrates and all components fabricated on them to conform to the various surfaces of the body. Medical implants that are to be placed in a brain vessel, for example, are expected to last a lifetime, so all materials and device components must be designed to withstand millions of stress cycles. NiTi SMAs are biocompatible, meaning they do not degrade in the patient's body or release harmful substances. In addition, some SMAs can be superelastic and deformed without fatigue.

From the prior art, a metamechanical auxetic design with island structure areas and interconnections, corresponding to the small interconnected island structure areas electrically connected to each other claimed herein, for functional device integration is available only from US 2020/0 144 431 A1. Unlike the referenced printed document, the auxetic web structure or field structure according to the invention features Archimedean spiral connections and/or self-similar fractal design connections. In the publication, only serpentine connections are used. In the auxetic web or field structure of the invention, the connections intersect the island structure faces at the same location and angle as the previous rigid connectors intersect the auxetic framework. No information on the angle of intersection can be found in the aforementioned printed material. Thus, this seems to have only a subordinate significance there at best, or actually no significance at all. Nevertheless, the cutting angle is of central importance for the auxetic web or field structure according to the invention.

The auxetic web structure or field structure according to the invention overcomes the prejudice that cantilevered metal joints are not mechanically robust enough to be free-standing web structures by using shape memory alloy. It is also possible to fabricate an array of different functional devices that are distributed over a plurality of island structure areas, which in turn are electrically connected to each other via the webs.

Each uniquely modified auxetic geometry offers a tradeoff of mechanical flexibility, stretchability, deformability, and areal density, and collectively offers a wealth of possible adaptable shapes that could be used to design next-generation medical devices.

In particular, the shape memory alloy may be NiTi. SMAs are used here as auxetic backbones for medical implants, as some NiTi-based biocompatible SMAs have already shown in trials that they can meet cycling requirements. The SMA can also be one of the NiTi alloys such as TiNiCu, TiNiCuCo, TiNiHf and the like.

Further, the electronic components may be selected from various elements, such as piezoelectric element, energy storage device, antenna structure, prefabricated microprocessors. Examples of active electronics that could be integrated on the island structure areas and/or backbone of the auxetic structure include: Electrodes, sensors (chemical, biological, electrical, magnetic, mechanical), actuators, interconnects, capacitors, LEDs, batteries and solar cells for energy storage, energy harvesters, and antennas. These components can be formed from essentially any material that is currently compatible with advanced micromachining and deposition techniques. Examples of materials include metals, oxides, dielectrics, perovskites, semiconductors, polymers, ceramics, piezoelectric, pyroelectric, magnetic, ferroelectric, and magnetostrictive materials.

In addition, shape memory alloy auxetic structures could be functionalized with other types of shape memory alloy actuator devices.

In addition to auxetic SMAs providing patient-specific design for medical implants, functionalization provides a novel feature by enabling wireless transmission of vital patient data to the outside world.

Auxetic shape memory alloys coupled with another shape memory with a different transition temperature could be used to create self-folding auxetic structures.

Another feature is that the shape memory alloys used here are electrically conductive materials with conductivities only ˜1 order of magnitude worse than conventional metals, such as platinum, copper, and gold. The electrical conductivity of SMAs is similar to that of liquid metals, which are considered one of the most ductile metals. The main challenge with liquid metals and conventional thin-film metals is that they must be integrated into an elastomeric substrate. The relatively high conductivity of the shape memory alloy coupled with its mechanical robustness allows the freestanding substrate to serve as a ground electrode to connect all active electronic components. Components can be fabricated on individual island structure areas and then electrically connected through a metallic SMA substrate. The combined strength of the SMA material and the auxetic geometric structure results in low stresses under applied loading throughout the auxetic structure. Therefore, the entire auxetic backbone structure could be designed to accommodate other thin film material traces and even potentially complete devices.

Thus, it is also possible to provide the auxetic web structure or field structure formed as a common ground electrode to improve the prior art.

Alternatively or even complementarily, at least two conductive tracks can be provided side by side spaced apart and/or one above the other each with an insulation layer between the metallic auxetic structure and the conductive track.

The immediate advantage is that a second metallic path can be insulated and in particular spaced from the ground electrode via a web in a fairly arbitrary manner, for example by a comparatively thicker dielectric layer between the two. In other words, one can provide a plurality of well-conducting paths for voltage signals via each of the individual connections/bridges of the scaffold structure, with at least one path existing that can withstand a predeterminable maximum voltage without damaging the implant. This expands the design capabilities of such a scaffold structure and its electronic population, ultimately the final functionality of a flexible electronic implant.

In one embodiment, a covering insulation layer and/or further insulation layers may be provided at least above the electrical connection.

Further, the insulating layer and/or further insulating layers of insulated conductive traces may preferably comprise a non-conductive oxide. Such a material may also be applied by means of the methods set forth in the present disclosure.

Furthermore, also in a preferred embodiment, the implantable structure may comprise, at least in sections, a biocompatible polymer as an outer layer.

In a particularly preferred embodiment, a layer, in particular a biocompatible layer, is applied to the layers and components provided at least on the auxetic metallic SMA structure. In one embodiment, an auxetic metallic SMA structure can remain free of an overlay in this case, since biocompatible material is used for this purpose.

Preferably, the structure aspect ratio, i.e. structure width to structure thickness, of the conductor track can be in the range of 0.5 to 1 at least in the region of the webs or the web structure.

Further, the structure thickness of the conductive path can be in the range of ˜10 nm up to 200 μm. For medical implants, the target thickness of the overall structure may well be in the range of ˜30-50 μm. However, for a wearable e-tattoo, conductive traces up to 200 μm would be possible since there is no spatial requirement for the component size.

A possible fabrication process of an implantable structure made of a biocompatible material has the following steps:
Fabricating an auxetic web structure or field structure having island structure areas and interconnections, wherein open spaces are present between the individual island structure areas and the interconnections form a web structure or field structure with the island structure areas, wherein at least individual island structure areas for receiving electronics or individual island structure areas equipped with electronics are formed and at least these island structure areas are not or not substantially bendable under the influence of force on the auxetic web or field structure and/or are not or only insubstantially variable in size, wherein for this purpose the biocompatible material is selected from a metallic shape memory alloy and the auxetic structure is formed from a metallic auxetic structure,
producing electronics and/or electronic components distributed on at least two island structure areas and electrically connected to one another, and an electrical connection between the electronics and/or the electronic components, this electrical connection being made by producing at least one conductor track electrically insulated from the metallic auxetic structure on at least one web structure of the metallic auxetic structure.

The at least one conductive track and/or the at least one insulating layer and/or the electronics and/or the electronic components on the auxetic structure can be particularly preferably sputtered on and/or also preferably applied using a further thin film technique, sol-gel technique or a chemical or physical deposition technique.

Further, the at least one conductor track and/or the at least one insulating layer can be applied together with the electronics and/or the electronic components on the auxetic structure. It is quite possible to perform corresponding steps together and thus achieve synergistic advantages in design and manufacturing.

By modifying traditional auxetic designs according to the invention, it becomes possible to integrate low-stress island structure areas into the basic framework of auxetic structures. These island structure areas are subjected to small stresses when subjected to mechanical loading, such as crimping or stretching. The island structure areas can be sized to accommodate any type of traditional rigid electronic MEMS or even NEMS components.

The use of shape memory alloys offers a great advantage in feature combination, as the auxetic structure can self-expand to extremely large volumes through a stress- or temperature-induced solid-to-solid phase transformation.

The extent of radial volume expansion is further increased by replacing the rigid connection points of the island structure faces with stretchable intermediate connections, i.e. Archimedean spiral connections and/or self-similar fractal design connections.

The implementation of shape memory alloys as the backbone of the auxetic structure provides better mechanical properties for the device/device, i.e., longer device/device lifetime. The shape memory effect is key to making it possible for the modified auxetic substrate to accommodate a high residual stress, making the medical implant resistant to deformation. Adding functionalized island structure areas increases the capabilities of the device by adding elements such as pressure sensors, strain sensors and more. The body's inflammatory response to device implantation could also be reduced by direct local drug release in one embodiment. Such a medical implant thus allows direct local delivery of drugs to the intended site in the body, which generally reduces the side effects of drugs administered by conventional means (e.g., orally).

In principle, SMAs can be crimped, i.e., folded to a small volume (without bending the islet structural surfaces) and unfold inside the patient even at body temperature. This allows, among other things, minimally invasive positioning in endoscopic surgery.

A preferred process for fabrication is based on fabricating all device components with MEMS processing techniques [micro-/nanoelectromechanical systems (MEMS/NEMS)] on an amorphous auxetic SMA substrate using a process published in U.S. Pat. No. 8,758,636 B2 (also referred to as Lima et al.). The entire composite is then formed into a complex 3D shape by annealing (>450° C.) to crystallize the SMA. The useful shapes envisioned here are a 3D cylindrical tube for stents and a 3D sphere or hemisphere for intrasaccular aneurysm devices that can be squeezed to sufficiently small dimensions to allow device insertion through a microcatheter. This feature would allow the devices to be used through endovascular treatments that are noninvasive and safer for the patient, i.e., shorter surgical time, less exposure to X-ray and MRI radiation. The process can be applied to pattern amorphous shape memory alloy materials into the desired 2D osseous structures. This process enables high geometric precision with feature sizes as small as 10 microns using photolithography, sputtering, and chemical wet-etch release processes. Because the SMA can be deposited amorphously with low surface roughness, they can be further processed up to any temperature (<800° C.), making the SMA substrate compatible with most modern MEMS processes. State-of-the-art substrates currently used in implants and in stretchable devices, such as polymers and elastomers, cannot handle these high temperatures (<200° C.).

While previously known commercial stents can contract and deploy in the body, most designs contain a somewhat rigid backbone that cannot conform to the shape of the artery. Auxetic stent structures would be superior to known stents because they can become patient-specific and adapt to any type of complex geometric surface, which is necessary because each patient's cerebral vessels, arteries, and aneurysms have different shapes and sizes. State-of-the-art auxetic stent structures have higher radial forces at the stent circumference, as much as 10 times higher than current stent designs. In addition, current stent designs do not provide large island structure areas that could be integrated or even functionalized with active and passive devices.

As previously illustrated, the invention described herein can serve as a platform for functionalizing any type of electronic device on virtually any type of complex geometric surface (from the macro to the nanoscale) using, in particular, auxetic shape memory alloy substrates.

Each uniquely modified auxetic geometry offers a tradeoff of mechanical flexibility, stretchability, deformability, and areal density, and overall offers a wealth of possible adaptable shapes that could be useful for designing the next generation of stretchable electronics. Most stretchable applications have similar goals: Maximizing stretchability and maximizing area coverage. i.e., enabling more functional areas for device integration.

A major advantage of the novel stretchable auxetic design is that all designs under applied tensile/compressive stress provide large island structure areas with low stress suitable to accommodate essentially any type of MEMS component. This key property can theoretically enable the co-integration of various electronic components, such as batteries, antennas, sensors, actuators, solar cells, and the like, through the substrate or a more conductive electrode deposited on the stretchable auxetic substrate. Semiconductor devices could be patterned directly into the self-similar and/or fractal and/or Archimedean interconnect shape to create deformable semiconductor arrays using similar methods as described in US 2020/0 144 431 A1.

The large island structure areas in the designs disclosed herein are subject to low stresses under applied strains that can accommodate electronic components for piezoelectric energy harvesters and semiconductor devices such as displays and solar cells, and for medical implants.

In a preferred embodiment, the structure may further have a self-similar fractal design in its entirety or in sections to improve the compressibility and extensibility of the auxetic structure. In another embodiment, the arranged (or bar-shaped) auxetic structure may have Archimedean spiral connections in its entirety or in sections.

In particular, the Poisson's ratio of the entire modified auxetic structure is negative, meaning that the structure expands under uniaxial tensile loading in the perpendicular direction. The negative Poisson's ratio of auxetic structures is advantageous for stretchable electronics because the structures can conform to any curved surface regardless of material and thickness. Modem stretchable microelectronic mechanical systems (MEMS) are usually based on wavelike cross-linked thin-film metals and island bridge layouts with thin-film serpentine interconnects. In addition, when stretched, the stretchable electronics should simultaneously maintain a large areal density for device integration.

Other improved mechanical properties generally offered by auxetic structures include high indentation/impact resistance, reduced fatigue crack propagation, greater toughness and modulus strength, and vibration damping. Because of these properties, the stretchable auxetic structures disclosed here are of potential interest for military and civilian applications such as bulletproof vests and impact-resistant vehicles and ships.

Electronic devices to be attached to the exterior of architectural buildings, vehicles, ships, aircraft, or inside missile ships also need to be extremely resistant to deformation. Many of these applications would benefit from stretchable auxetic substrates, as the structures presented here can be fabricated in a 2D film and bend around 3D curved surfaces such as spheres, curved tubes, and cylinders.

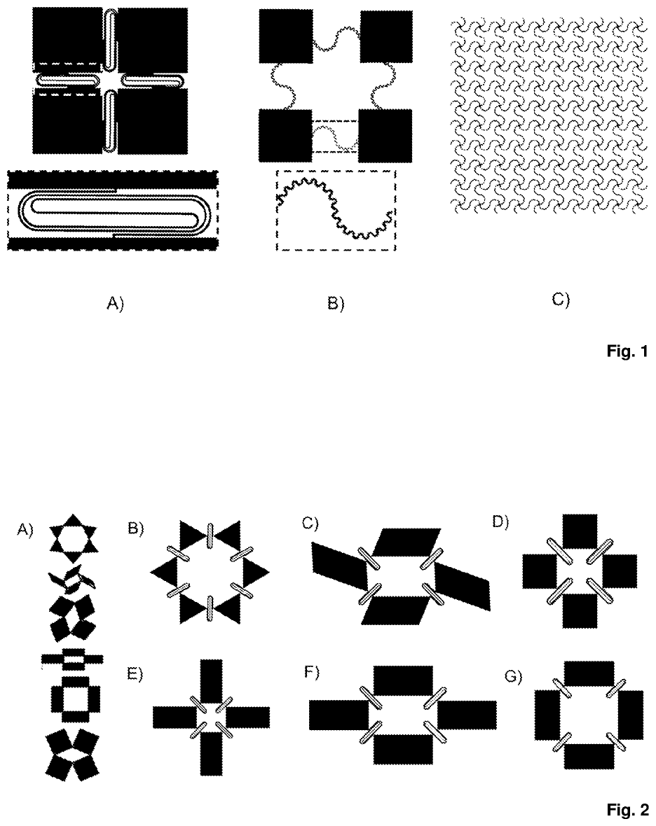

A difference and key feature of the invention can be seen in the fact that conventional stretchable electronic structures configured in the island interconnect format have a Poisson's ratio of zero as they connect at the center of the island, as shown in the Archimedean spiral interconnect in FIG. 1A and the fractal horseshoe serpentine interconnect in FIG. 1B.

A key difference and a key feature of the invention can also be seen in the fact that the prior art designs also do not connect island structure areas with Archimedean spiral connections and/or self-similar fractal design connections, thus providing sufficient space and area for future innovation through MEMS sensor/actuator integration due to the large island structure areas. As can be seen in FIG. 1C, prior art wave-shaped mesh structures do not provide sufficient space for sensor integration.

Another aspect of the present invention is to improve the radial expandability of the auxetic structure by replacing the rigid hinge connection points of the rotating auxetic polygonal structures, or unit cells, with classical Archimedean spirals and/or self-similar fractal design connections. This concept is made clear by introducing a new way of representation, as shown in the figures with the examples of Archimedean spiral connections. By mechanically shaping the geometric properties of the auxetic structure, i.e., surface density, Archimedean spiral radius, etc., it is possible to tune the flexibility/extensibility of the structure as well as the bulk modulus, shear modulus, elastic modulus, and Poisson's ratio of the auxetic substrate. The freedom of geometric design also enables the development of stretchable auxetic substrates with high areal coverage, which could enable higher efficiency devices in the future.

There are several auxetic mechanical metamaterials in the literature that can be modified to contain low-stress island structure areas. Some examples include honeycomb, chiral, re-entrant, origami-based metamaterials, kirigami-based metamaterials, keyed-brick structures, slotted-perforation structures, rotating rigid structures, and hierarchical auxetic systems.

The auxetic structure according to the invention requires a significant modification of the traditional auxetic structures from the literature by replacing their rigid or rotating connection points with stretchable connections. The meandering wave-like connections (winding path structures) are formed from known stretchable connections, with one embodiment of the invention showing auxetic structures with stretchable connections from Archimedean spiral structures. A second embodiment shows auxetic structures with stretchable fractal connections, and a third embodiment shows auxetic wavy mesh design. To ensure that the modified structure maintains its auxetic behavior, the stretchable connection must intersect the auxetic framework at the same location and angle as the previous rigid connectors. Replacing the rigid connectors with extensible connectors greatly improves the mechanical properties of the overall auxetic structure and allows extreme extensibility and compressibility of the overall structure.

The invention is accomplished by creating an array of periodic island structural surfaces that are auxetically arranged, and then connecting the island structural surfaces with periodic stretchable intermediate connections (e.g., meandering fractal design connections or Archimedean spirals). The resulting stretchable auxetic structures can enable any material to become extremely resistant to applied deformation.

The island structure areas in the structure experience low stresses under applied strain, making them suitable for large area integration of functional electronic MEMS devices. This property is necessary to enable the integration of wireless transmission antennas, energy harvesting devices, energy storage devices, and virtually any type of sensor or actuator component into medical components and wearable devices.

The design of the geometry of the stretchable interconnects (i.e., amplitude, wavelength, thickness, and width) allows for the development of a trade-off between maximum areal density and stretchability. Large area conformal stretchable auxetic substrates would enable MEMS/NEMS devices with higher efficiency.

Specifically, all of the above applications require that the substrate material be compatible with traditional 2D MEMS wafer-based processing techniques.

The disclosed stretchable auxetic structures will have numerous applications as substrate/backbone structures in the fields of stretchable electronics, wearable electronics, shock resistance, energy absorbing materials, architecture, space exploration, and implantable medical technologies. When the structure is used as a substrate, any material coated onto a stretchable auxetic substrate benefits from the enhanced mechanical properties provided by the underlying structure.

The archetypal design further presented here allows for extreme flexibility and stretchability within any type of device, which will be important for next-generation medical applications such as smart stents, intrasaccular aneurysm devices, drug-delivery implants, and wearable biosensors.

Shape memory alloys (SMAs) structured in auxetic geometries offer superior mechanical properties that can enable the design of high-performance novel medical implants with enhanced mechanical flexibility and ductility. SMAs can achieve large intrinsic strains of up to 8% through thermal or stress-induced phase transformation. In addition, SMAs are used in the present invention because they can be microstructurally designed to undergo (thermal) stress-induced (mechanical) phase transformation for 10 million cycles without changing their functional fatigue properties. With these properties, the life of a medical device/device can be increased, making the device safer overall for patients.

The invention also has potential applications in the field of wearable electronics. For example, electronic tattoos and other substrates worn directly on the skin must conform naturally to the skin as well as be reversibly stretchable over very long distances for thousands or millions of cycles. Thanks to synclastic bending, auxetic SMAs can conform to the surface of the skin or substrate better than stretchable current designs, while providing greater area coverage and better mechanical performance than current serpentine grid designs. This could be useful in applications requiring bio-electrodes (skin sensors), such as electrocardiogram and magneto-cardiogram sensors, which require a perfect interface with the skin. These applications benefit from the discreet electronic conductivity offered by metallic SMAs. The superior mechanical properties of the SMA material and auxetic geometries would benefit many applications related to stretchable and wearable electronics and soft robotics.

The invention is described below with reference to the accompanying figures in the figure description, which is intended to explain the invention and is not necessarily limiting. Showing:

FIG. 1 overview of stretchable geometries known in the prior art having. Similarities to the present invention;

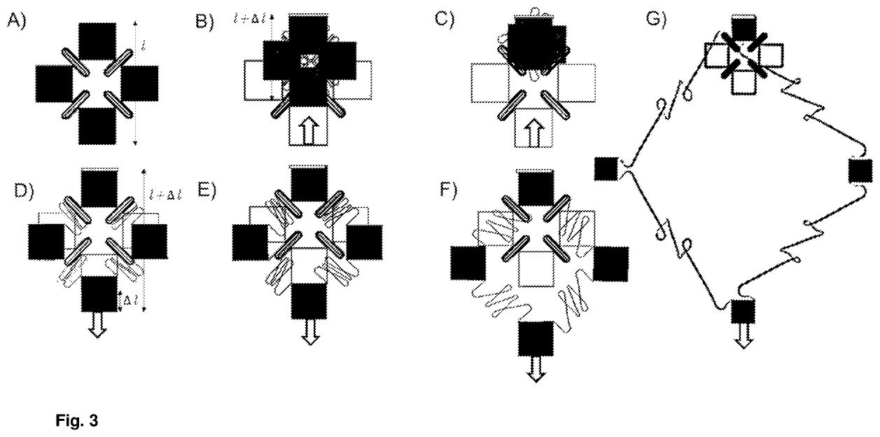

FIG. 2 rotating rigid auxetic structures according to the prior art (FIG. 2A) and embodiments of Rotating Archimedean polygons according to the invention (FIGS. 2B-2G);

FIG. 3 finite element simulations showing the improved ductility and compressibility of an exemplary rotating Archimedean square structure in accordance with the Archimedean square structure according to the invention (FIGS. 3A-3G);

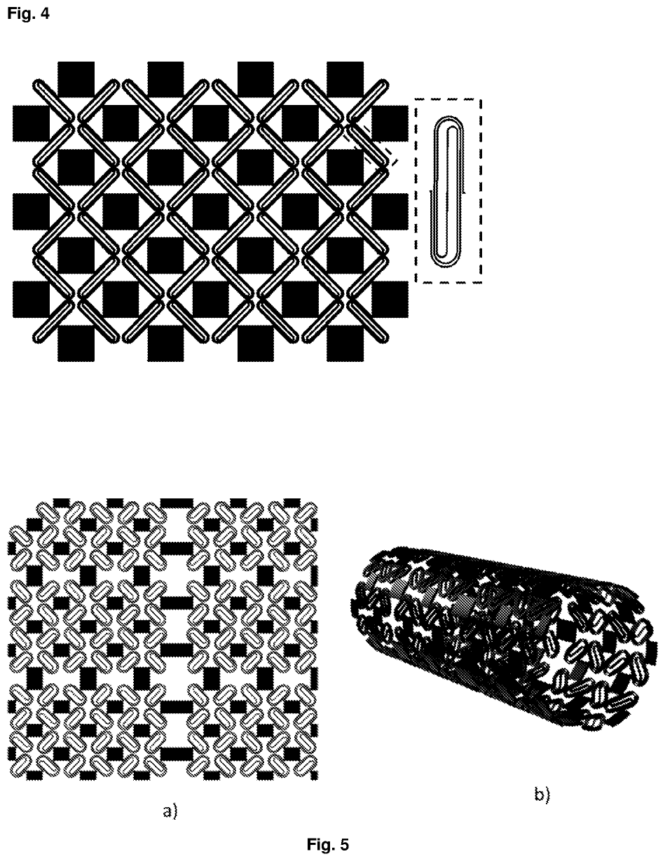

FIG. 4 an embodiment of a rotating Archimedean square structure according to the invention. square arrangement (detail—a single unit of the Archimedean spiral connection);

FIG. 5 an example of a rotating Archimedean rectangular structure according to the invention rectangular structure formed as a 2D plate (FIG. 5A) and as a 3D cylinder (FIG. 5B);

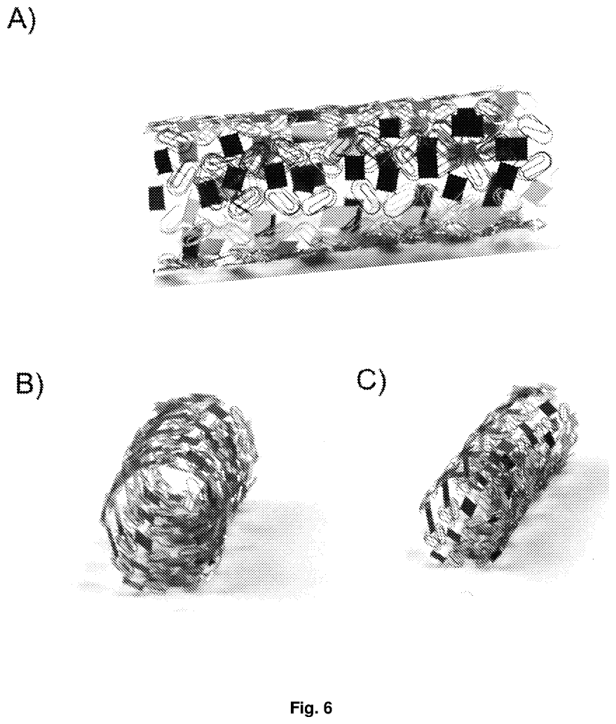

FIG. 6 an experimental realization of a rotating Archimedean rectangular structure according to the Archimedean rectangular structure in the form of a 3D cylinder with a diameter of a diameter of 5 mm and a length of 9 mm from different perspectives (FIGS. 6A-6C);

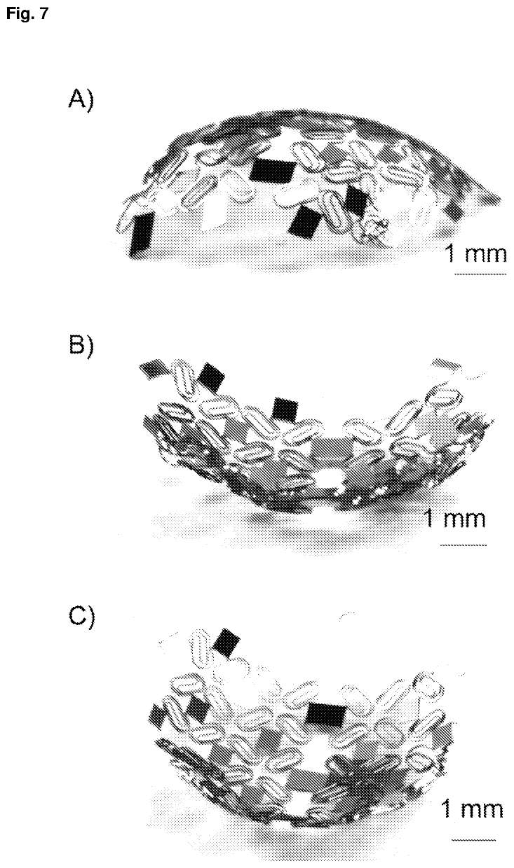

FIG. 7 an experimental realization of a rotating Archimedean rectangular structure shaped according to the invention. Archimedean rectangular structure formed into a 3D hemisphere with a radius of 2.5 mm from different perspectives (FIGS. 7A-7C);

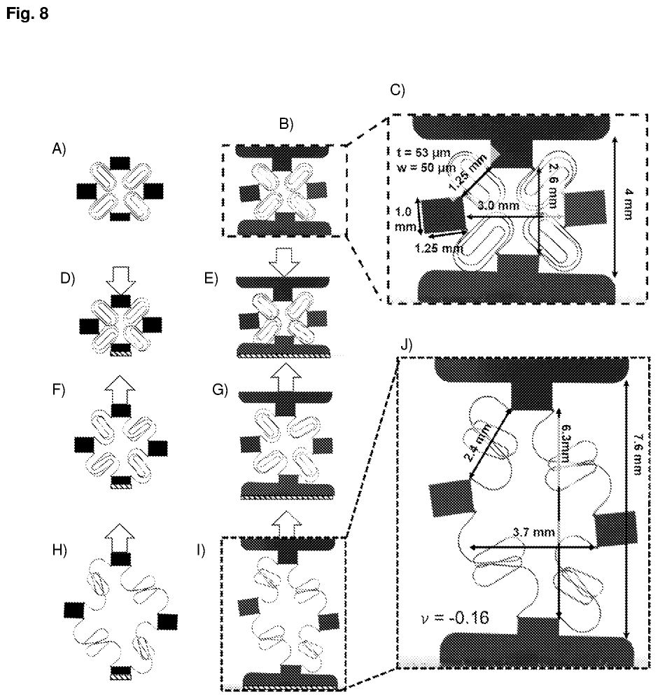

FIG. 8 finite element simulations and experimental comparison of a first exemplary rotating Archimedean rectangular unit cell according to the invention.unit cell, which shows a clear auxetic behavior in uniaxial tension and compression tests;

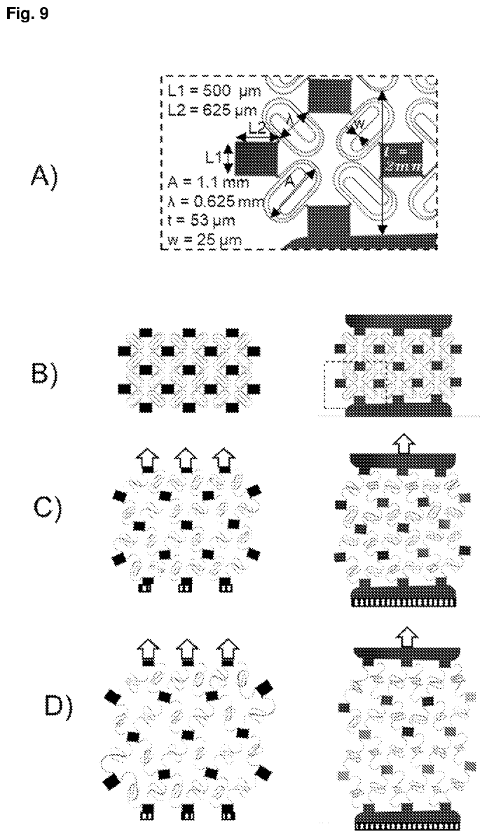

FIG. 9 finite element simulations and experimental comparison of a second example of a rotating Archimedean rectangular structure, which exhibits significant auxetic clear auxetic behavior;

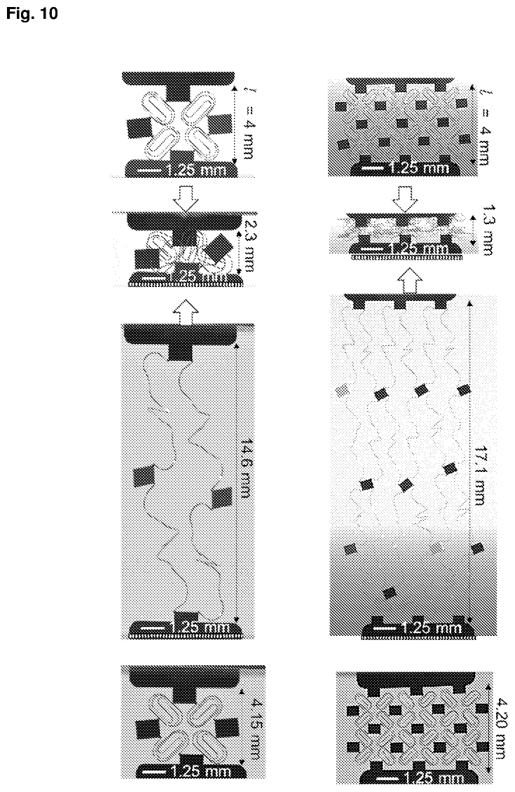

FIG. 10 a demonstration of elastic and superelastic recovery from enormous tensile and compressive loads of the embodiments shown in FIG. 8 and FIG. 9. Examples of embodiments based on rotating auxetic archimedean auxetic rectangular structures;

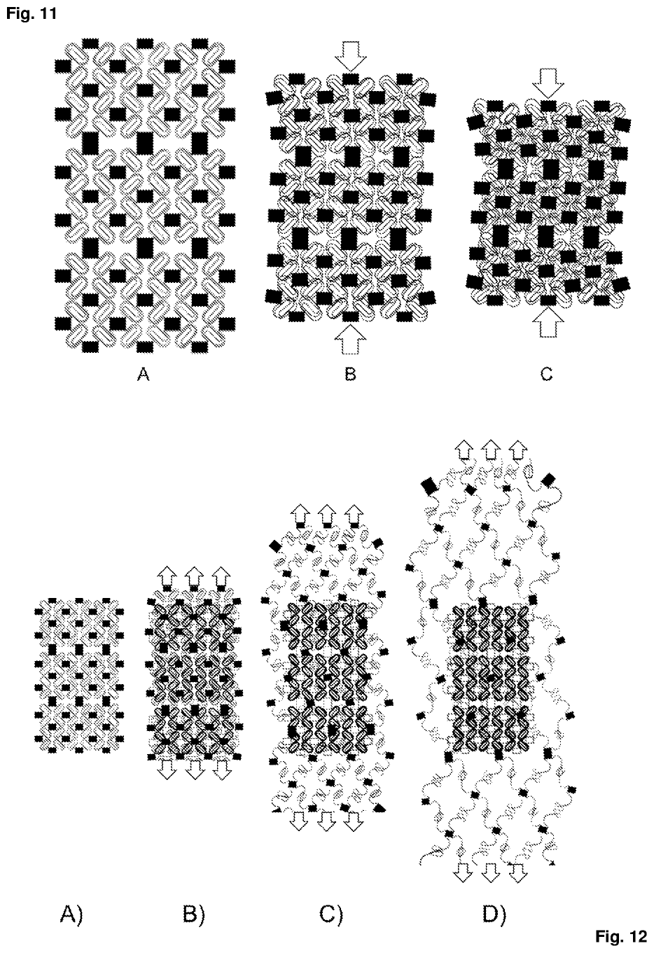

FIG. 11 finite element simulations of the enhanced compression in an exemplary rotating Archimedean auxetic rectangular structure according to the invention rectangular structure (FIGS. 11A-11C);

FIG. 12 finite element simulations of the extraordinary ductility in an exemplary rotating Archimedean rectangular structure according to the invention rectangular structure (FIGS. 12A-12D);

FIG. 13 an embodiment example of a rotating rectangular auxetic structure with second-order fractal horseshoe-serpentine connections as a 2D surface (FIG. 13A), and the rotating rectangular auxetic structure with second-order fractal horseshoe-serpentine connections in 3D cylindrical form (FIG. 13B);

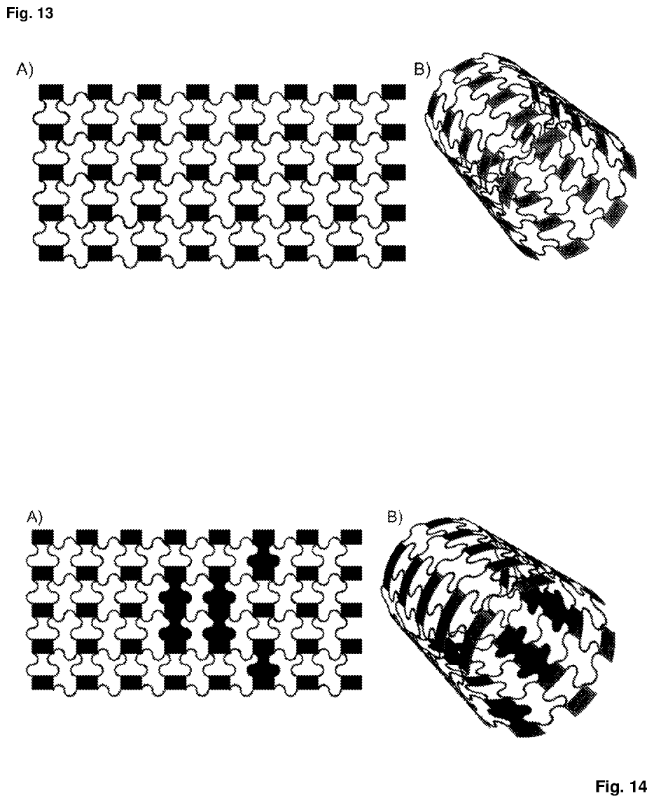

FIG. 14 an embodiment of a rotating rectangular auxetic structure with second-order fractal horseshoe-serpentine connections and island structure areas of different sizes as a 2D surface (FIG. 14A) and, the rotating rectangular auxetic structure with second-order fractal horseshoe-serpentine connections and island structure areas of different sizes formed into a 3D cylindrical geometry (FIG. 14B);

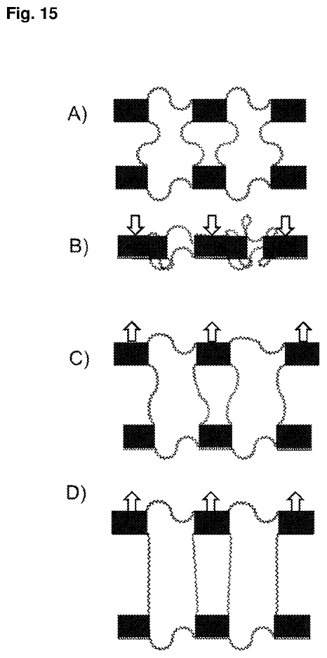

FIG. 15 finite element simulations demonstrating the auxetic behavior of the example structure according to the invention shown in FIG. 13 based on a rotating rectangular auxetic structure with second-order fractal horseshoe-serpentine connections during expansion and compression (FIGS. 15A-15D);

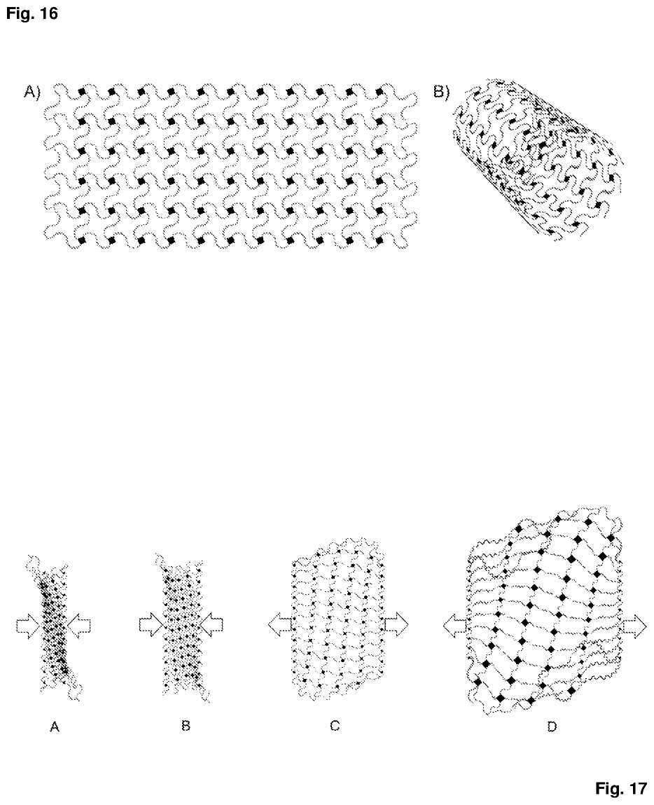

FIG. 16 an example of 2D lattice design with a square lattice geometry using second-order fractal horseshoe-serpentine connections and square island structure areas as 2D surface (FIG. 16A), and the 2D lattice design with a square lattice geometry using second-order fractal horseshoe-serpentine connections and square island structure areas formed into a 3D cylindrical geometry (FIG. 16B);

FIG. 17 finite element simulations showing auxetic behavior with enhanced expansion and compression in the embodiment of a 2D lattice design structure shown in FIG. 16 using second-order fractal horseshoe-serpentine connections and square island structure faces (FIGS. 17A-17D);

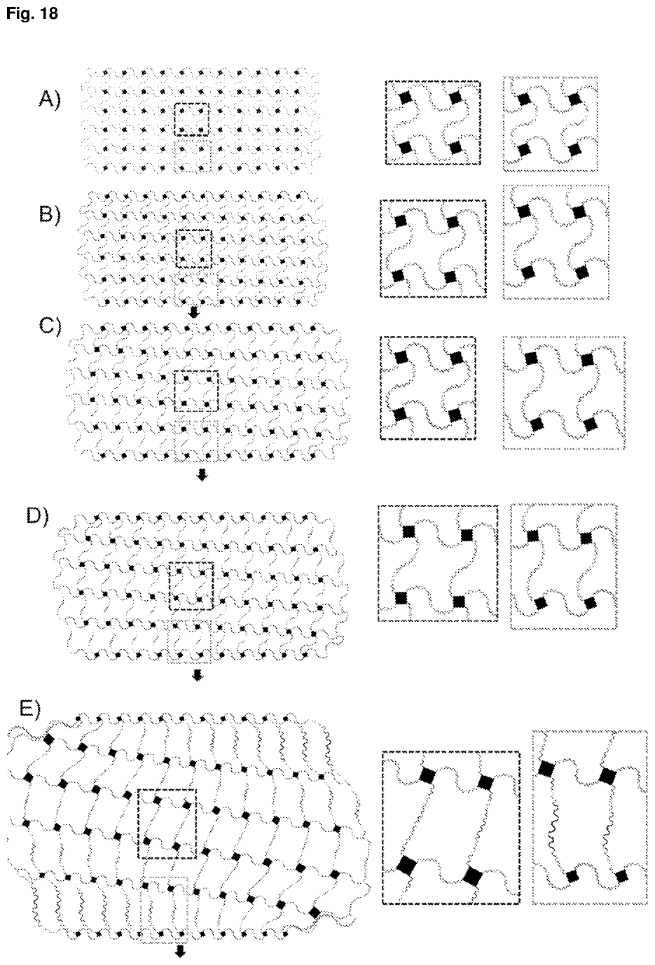

FIG. 18 finite element simulations showing auxetic behavior with exceptional ductility for the design example shown in FIG. 16, where uniaxial strain is applied from one side of the structure and the other boundary is held as a fixed constraint (FIGS. 18A-18E);

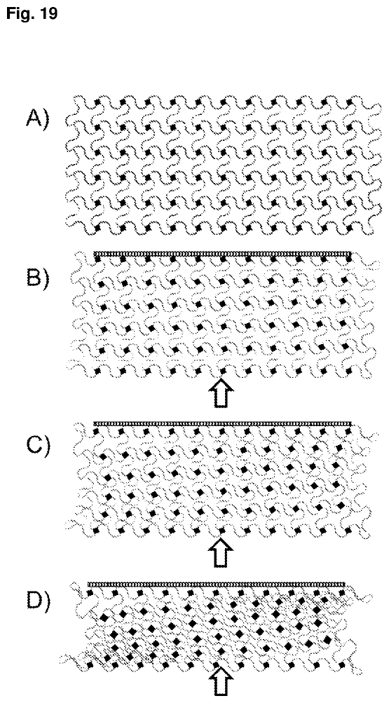

FIG. 19 finite element simulations showing auxetic behavior with increased compressibility for the embodiment example shown in FIG. 16, where uniaxial compressive strain is applied from one side of the structure and the other boundary is held as a fixed constraint;

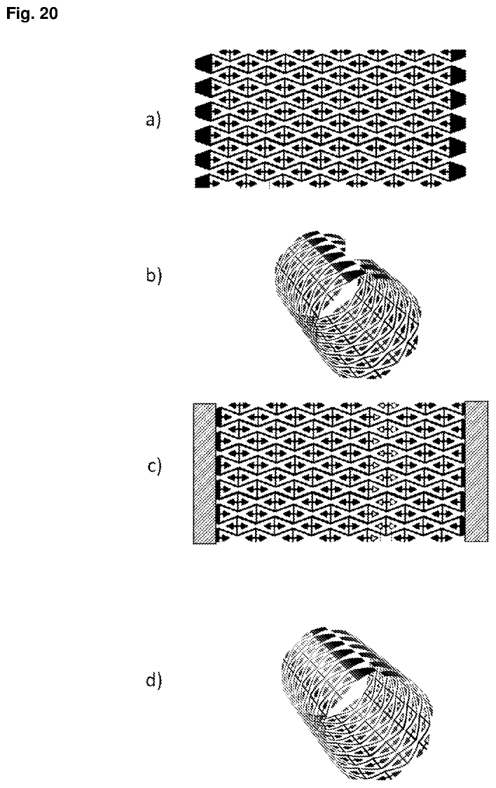

FIG. 20 an exemplary flowchart showing steps a)-d) to illustrate the 3D fabrication of a functionalized shape memory alloy implant structure according to the invention;

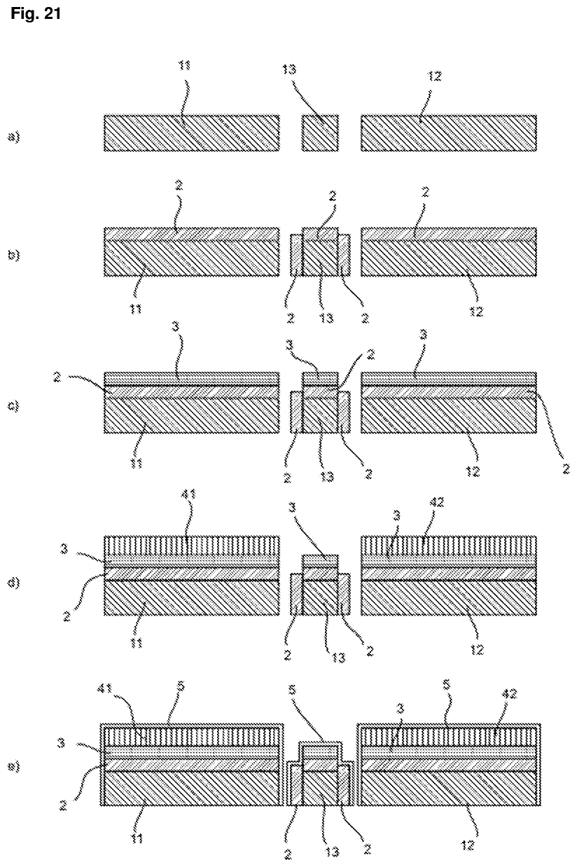

FIG. 21 an example schematic flowchart showing device cross-section steps a)-e) for functionalizing an implantable auxetic structure according to the invention;

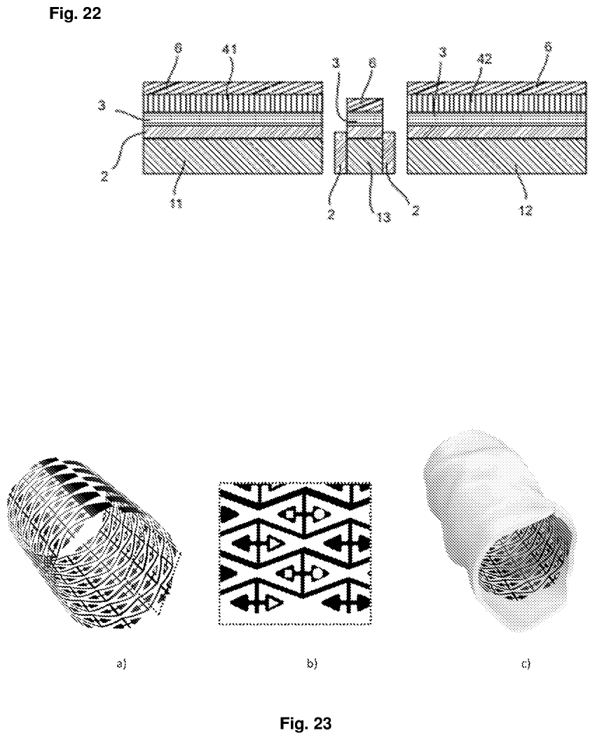

FIG. 22 an example corresponding to FIG. 21 section d), supplemented by at least one additional electrode;

FIG. 23 a first embodiment example of a functionalized implantable structure (smart stent) according to the invention in 3D tubular form (FIG. 23A), in section the unit cell of the functional auxetic structure with three different device components (FIG. 23B) and embedded in a blood vessel or artery (FIG. 23C);

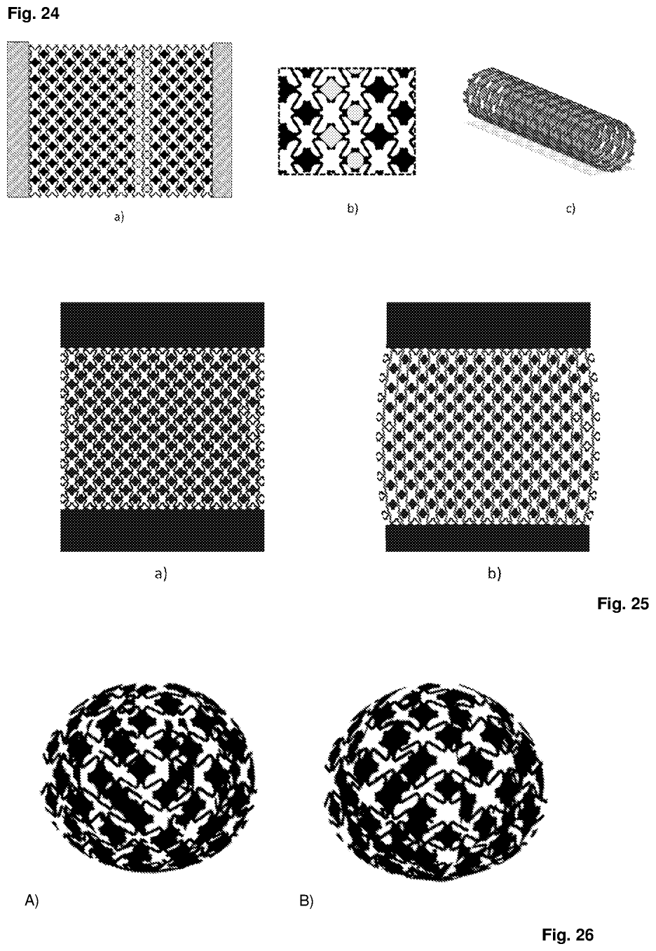

FIG. 24 a second embodiment example of an “S-shaped island structure area” auxetic geometry modified to provide island structure areas for device integration from conventional “S-shaped” auxetic structures, shown as a 2D surface (FIG. 24A), a section (FIG. 24B), and a 3D smart stent (FIG. 24C);

FIG. 25 finite element simulations of the embodiment of an “S-shaped island structure area” auxetic structure from FIG. 24 with the structure in equilibrium (FIG. 25A) and under uniaxial tension (FIG. 25B); and

FIG. 26 illustration of the synclastic bending behavior of an “S-shaped island structure area” around a 3D sphere, showing that it could be used as an intrasaccular device.

FIG. 1 shows examples of the state of the art in stretchable electronics. FIG. 1A shows a traditional island interconnect structure with Archimedean connections according to Jiang et al. ARCHIMEDEAN SPIRAL DESIGN FOR DEFORMABLE ELECTRONICS U.S. Pat. No. 10,660,200 B2 (2020). FIG. 1B shows a traditional island interconnect structure with second-order fractal horseshoe-serpentine interconnects, and FIG. 1C shows a 2D wave network with horseshoe-shaped building blocks arranged in a square lattice geometry. Designs according to FIGS. 1B and 1C are known from the Rogers et al. Self-similar and fractal design for stretchable electronics U.S. Pat. No. 10,192,830 B2 (2019).

As previously shown, prior art stretchable island interconnects are typically connected at the center of the islands, resulting in a Poisson's ratio of zero under uniaxial loading. The structures must be stretched biaxially (in the x and y directions) to achieve expansion, or compressed biaxially to achieve a fully compressed structure. In uniaxial tensile tests (in the y-direction only), the structures exhibit a positive or zero Poisson's ratio. The 2D corrugated network proposed in FIG. 1C is known to exhibit auxetic properties at uniaxial strain. The major drawback of this design is that large area islands are not available for integration of functional components.

FIG. 2 shows:
A) Examples of traditional prior art auxetic structures based on rotating rigid structures for example known from Saxena, Krishna Kumar, Raj Das, and Emilio P. Calius. “Three decades of auxetics research-materials with negative Poisson's ratio: a review.” Advanced Engineering Materials 18.11 (2016): 1847-1870
and further in B) to G) embodiments of auxetic structures with increased ductility using Archimedean connections according to the invention, namely:
B) rotating Archimedean triangle
C) rotating Archimedean parallelogram
D) rotating Archimedean square
E) rotating Archimedean rectangle variant 1
F) rotating Archimedean rectangle variant 2
G) rotating Archimedean rectangle variant 3.

The structures presented in this invention provide an extremely compliant and deformable substrate that allows any material to become flexible, stretchable, and conformable. FIG. 2A shows examples of traditional rotating rigid polygon auxetic geometries known in the prior art. FIGS. 2B-2G show modifications of the rotating rigid auxetic structures of the invention with Archimedean spiral connections to provide improved extensibility and compressibility. Here, the stretchable Archimedean spiral is connected to the island structure faces at the same angle at which the island structure faces were previously connected in a traditional rotating polygon structure.

Wearable devices that are intended to be worn directly on the skin must be able to adapt to extreme motion and the resulting changes in body curvature radius. Stretchable auxetic structures must be able to freely expand and contract to accommodate abrupt changes in deformation.

The auxetic behavior is shown in the figures below for extensible auxetic structures based on rotating squares of the same size and rotating rectangles of different sizes.

Finite element analysis (FEM) using COMSOL Multiphysics 5.6 is used in some of the figures below to show the auxetic behavior of the structures according to the invention under a uniaxial compressive force (compressed) and a uniaxial tensile force (stretched). The illustrated structure according to the invention has a negative Poisson's ratio, as shown by the volume expansion in the perpendicular direction under uniaxial displacement. The maximum extensibility of the unit cell depends on the design of the exemplary Archimedean spiral connection used.

For all FEM simulation results presented, copper was used as the material, assuming a Young's modulus of E=119 GPa, a Poisson's ratio of 0.34, and a density of 8940 kg/m3.

Analysis of the von Mises stress distribution after applying strain to the unit cell confirms low stresses on the large periodic island structural surfaces, as the stress is concentrated and distributed throughout the ductile joint for all modeled structures.

FIG. 3 shows FEM results illustrating the extent of improved compressibility and ductility for rotating square auxetic structures with Archimedean connections according to the invention. The island structure areas are 3.2 mm×3.2 mm. The Archimedean spiral connections have a peak-to-peak amplitude of 5 mm, a wavelength of 1.25 mm, a width of 50 μm, and a thickness of 50 μm. Macroscopic uniaxial strain is applied to the lowest edge of the structure in the direction indicated by the arrow.

FIG. 3A shows the structure undeformed at equilibrium with a neutral end-to-end distance=1. The amount of macroscopic compressive or tensile stress is defined as change in length (Δl)/distance from end to end at equilibrium (macroscopic stress %=Δl/l*100%).

FIG. 3B shows a black rectangle with a white area on one side to represent a “fixed constraint” on one edge of the square, while a uniaxial deflection is applied to the opposite square. The large arrow indicates that the direction of the applied force is a compressive force (in the direction of the fixed constraint). This figure shows the biaxial compressive behavior of the structure at a uniaxial compressive load of −30%. The magnitude of the macroscopic compressive or tensile load is defined as (Δl/l).

FIG. 3C shows that the rotating square Archimedean structure deforms out-of-plane after an applied compressive strain of −60%, so that the out-of-plane deformation causes the structure's island structure faces to stack almost on top of each other.

FIGS. 3D-3G show the auxetic structure according to the invention at various macroscopic uniaxial strain levels (˜10%-300%). Indicated by the arrow pointing away from the fixed restraint, one end of the auxetic structure is pulled under uniaxial tension. The structure clearly exhibits a negative Poisson's ratio, represented by an extension in the perpendicular direction of the applied tensile force.

FIG. 3G shows the expansion of the structure by four times its original area (300% of the total expansion of the original length).

The development of an array based on the presented exemplary structure according to the invention would enable a highly deformable 2D electronic device that can conform to essentially any type of concave or convex 3D surface.

FIG. 4 shows an example of a rotating square Archimedean arrangement. Here, the detail shows a single unit of the Archimedean spiral linkage.

An array of a rotating square Archimedean polygonal structure is suitable as a substrate for an implantable medical device, as this would allow improved extensibility and compressibility of the structure to meet crimp requirements to be inserted into the body through a microcatheter For a given stent circumference, traditional auxetic stent structures provide 10 times higher radial forces than current designs (as known, for example, from Dolla, William Jacob S., Brian A. Fricke, and Bryan R. Becker. “Structural and drug diffusion models of conventional and auxetic drug-eluting stents.” (2007): 47-55.). The addition of tortuous bar structures/connections, also known as stretchable interconnectors, to the framework of auxetic structures reduces the bending stiffness of the overall structure, resulting in crushing and stretching under small applied forces. This means that medical devices based on stretchable auxetic structures are easier to crimp, maneuver, and deploy through a microcatheter.

FIG. 5 shows a different arrangement of the rotating Archimedean rectangular structure with different sized rectangles and an Archimedean spiral with a smaller tip-to-peak amplitude (2.2 mm) compared to the spiral shown in FIG. 4.

A major advantage of the stretchable auxetic structures presented here is that they can be fabricated planar in 2D and then conform to 3D surfaces. If the structure is made of an amorphous shape memory alloy, it is possible to fabricate, for example, a medical implant entirely using 2D MEMS techniques, and then to shape the shape memory material into an intricate 3D geometry by crystallization of the shape memory alloy. This concept is illustrated in FIG. 5B, where the 2D plate was formed into a 3D tubular (or cylindrical) shape.

FIG. 6 shows an experimental implementation of a rotating Archimedean auxetic SMA rectangular structure made of TiNiCu (thickness=27 m) according to the invention, which was brought into a 3D tubular (or cylindrical) shape with a radius of curvature of 2.5 mm and a length of 9 mm. The structure could be used as a smart stent for flow detour, with large island structure areas available for electronic integration.

FIG. 7 shows an experimental realization of a rotating Archimedean auxetic SMA rectangular structure made of TiNiCuCo (thickness=53 m) in a 3D hemispherical geometry with a radius of curvature of 2.5 mm according to the invention. This structure could be used as a functionalized intrasaccular aneurysm device.

FIG. 8 shows the improved distensibility and compressibility under uniaxial stretch of a unit cell with rotating Archimedean rectangular geometry (embodiment shown in FIG. 5). Uniaxial strain was applied to the top edge of the structure in the direction indicated by the arrow, while the bottom edge of the structure was held as a fixed restraint (indicated by a striped rectangle). The left column shows the results of finite element modeling of auxetic behavior using the material properties of copper (FIG. 8A, D, F, H). The middle column shows experimental results of a rotating Archimedean rectangle made from the superelastic shape memory alloy TiNiCuCo (FIG. 8B, E, G, I).

Shape memory alloys (SMAs) are known to have better mechanical properties than conventional metals such as copper. SMAs are the preferred substrate material for the structures presented in the present invention. The shape memory alloy NiTi is biocompatible and is already used in medical devices such as stents. TiNiCu and TiNiCuCo alloys are not biocompatible, but have already been shown to be ultra-low fatigue SMAs capable of cycling between 0% and 2% intrinsic strain for more than 10 million cycles (Chluba, Christoph, et al. “Ultralow-fatigue shape memory alloy films.” Science 348.6238 (2015): 1004-1007.). Novel stretchable auxetic structures were fabricated from ultra-low fatigue TiNiCuCo thin films (thickness=53 m) to provide proof of concept.

In FIGS. 8A, B), the finite element simulation (FIG. 8A) and experiment (FIG. 8B) show an example rotating Archimedean rectangular unit cell in equilibrium (the end-to-end distance (l) is 4 mm). The inset in the right column denotes the dimensions of the island structure areas (1.0 mm×1.25 mm) and the Archimedean spiral (wavelength (λ)=1.25 mm, peak-to-peak amplitude (A)=2.2 mm, width (w)=50 μm, thickness 53 μm) of the structure at equilibrium. In both the simulations and experiments, the outermost edges of the geometry at equilibrium are assumed to be 4 mm apart. The distance between the innermost edges of the rectangles is used as the initial value for calculating the Poisson's ratio (xequilibrium=3.0 mm, yequilibrium=2.6 mm).

In FIGS. 8D, E, a total uniaxial compressive strain of −10% is applied to the uppermost edge of the structure, resulting in contraction island structure areas in the perpendicular direction.

FIGS. 8F, G show a uniaxial tensile strain of 26.5% applied to the top edge of the structure, resulting in contraction island structure faces in the vertical direction.

Furthermore, FIGS. 8H, I show a uniaxial tensile strain of 90% on the top edge of the structure, resulting in an extension of the island structure faces in the vertical direction. With a displacement of 7.6 mm in the y-direction, the extensibility (total strain) of the entire unit cell structure is 90%. The inset shows that the value of total elongation is determined based on the vertical distance between the outer edges of the top and bottom squares. The distance between the innermost edges of the rectangles is used as the initial value for calculating the Poisson's ratio (xstretched=3.7 mm, ystretched=6.3 mm).

In FIGS. 8C, J, it is shown by respective expansion and compression in the perpendicular direction of the applied force that this rotating Archimedean rectangular structure is indeed auxetic. The structure was experimentally shown to be auxetic with a Poisson's ratio of −0.16, calculated by dividing the change in axial strain ((3.7 mm−3.0 mm)/3.0 mm) by the change in transverse strain ((6.3 mm−2.6 mm)/2 .mm). The values for this calculation use the internal spacing between the island structure faces, since the strain of the spirals is responsible for the expansion/compression of the entire structures. Non-uniform strain was observed in the four Archimedean spirals of the unit cell during the strain experiments, resulting in strains ranging from 101% to 189%. The non-uniform behavior in the Archimedean spirals could be due to 1) the angle of connection of the spiral with the island structure faces and 2) misalignment of the structure in the x-axis when tensile tests were performed parallel to the y-axis.

All dimensions and values shown in FIG. 8 are exemplary for the exemplary arrangement of the rotating Archimedean rectangular structure shown and should not be considered limiting for differently designed structures according to the invention.

FIG. 9 shows finite element simulations (FEM) and an experimental comparison of a second embodiment of a rotating Archimedean rectangular arrangement in which the arrangement is both auxetic and highly extensible. With the exception of the thickness (t=53 kim), all dimensions of the unit cell shown in FIG. 8 shown were reduced by a factor of 2 (the island structure areas are 0.5 mm×0.625 mm) and given an Archimedean spiral (wavelength (λ)=0.625 mm, peak-to-peak amplitude (A)=1.1 mm, width (w)=25 μm, thickness 53 μm) and then arranged into an array (3×2) to produce the array structure shown in FIG. 9. The simulation results in the left column use the material properties of copper, while the experimental results in the middle column again use the shape memory alloy TiNiCuCo.

The lower edge is a fixed constraint, while the upper edge undergoes a prescribed uniaxial displacement in the direction of the arrow.

FIG. 9A) shows the fabricated structure in equilibrium with all relevant dimensions given for the island structure faces (0.5 mm×0.625 mm) and the Archimedean spiral (w=25 μm, t=53 m, λ=0.625 mm, A=1.1 mm).

FIG. 9B) shows finite element simulation and experiment of the structure in equilibrium (0% applied strain).

FIG. 9C) shows FEM and experiment of the structure at uniaxial 62% tensile loading from the top edge.

In addition, FIG. 9D) shows FEM and experiment of the structure at a uniaxial tensile load of 102% from the top edge.

All dimensions and values shown in FIG. 9 are exemplary for the exemplary arrangement of the rotating Archimedean rectangular structure shown and should not be considered limiting for differently designed structures according to the invention.

FIG. 10 shows the experimental elastic recovery of giant strains and compressions of the geometries shown in FIG. 8 (rotating Archimedean rectangular unit cell) and FIG. 9 (rotating Archimedean rectangular arrangement). The elastic recovery of such large total strains is due to the combination of the Archimedean spiral and the superelastic effect known for TiNiCuCo shape memory alloys.

FIG. 10A) shows the rotating Archimedean rectangular unit cell structure (left column) and ordered structure (right column) in equilibrium (0% applied strain).

FIG. 10B) shows both structures at maximum tested compression of −47% (unit cell) and −67% (array).

FIG. 10C) depicts both structures at maximum tested strain of 265% (unit cell) and 330% (array). At this applied total strain, the maximum strain in the Archimedean spirals is 454% (unit cell) and 780% (array).

Superelastic recovery of elastic strain after removal of the applied force with extremely low total plastic strain to the structures of 3.75% (unit cell) and 5% (array) is shown in FIG. 10D).

The macroscopic yield strength of copper in the extensible auxetic geometries is determined when the yield stress exceeds 200 MPa or the first principal strain exceeds 2%. According to the simulation results, copper thin films with the same dimensions as the unit cell structures experimentally demonstrated in FIG. 10 can elastically uniaxially stretch the unit cell by only 15% (displacement of 0.6 mm), while TiNiCuCo can be elastically stretched by 265%. Similarly, copper can stretch elastically in the lattice structure to a maximum value of 112.5% (displacement of 4.08 mm), while TiNiCuCo can stretch elastically by 330%. Exceptional ductility was achieved in the shape memory alloy Archimedean spiral with almost complete elastic recovery from a strain of 780%. These significant results support the notion that shape memory alloys are a preferred material for stretchable electronic substrates fabricated using the structures disclosed in the present invention.

All dimensions and values shown in FIG. 10 are exemplary for the exemplary illustrated arrangement of the rotating Archimedean rectangular structure, and are not intended to be limiting for differently designed structures according to the invention.

For the illustration in FIGS. 11 and 12, the structure according to the invention shown in FIG. 9 is arranged again (1×3) to create a larger rotating Archimedean rectangular structure.

In FIG. 11, exemplary FEM of enhanced compression in a rotating rectangle with an Archimedean auxetic structure (FIGS. 11A (equilibrium)-9C (highly compressed)) are shown. For this purpose, a uniaxial compressive load is applied to the top and bottom edges of the structure in the directions indicated by the arrows.

FIG. 11A) shows the structure in equilibrium (0% total strain).

In FIG. 11B), the structure is shown under 10% compressive strain from the top edge and 10% compressive strain from the bottom edge, 20% total uniaxial compressive strain.

In FIG. 11C) shows the structure under 16.66% compressive stress from the top edge and 16.66% compressive stress from the bottom edge. This gives 33.33% uniaxial compressive stress in total.

FIG. 12 shows the simulation results of the auxetic behavior of the structure under uniaxial tension. It shows the uniaxial tensile loading at the top and bottom edges of the structure in the directions indicated by the arrows.

FIG. 12A) shows the structure at equilibrium (0% applied strain).

In FIG. 12B), the structure is under 10% compressive strain from the top edge and 10% compressive strain from the bottom edge. This gives 20% uniaxial compressive strain overall.

FIG. 12C) shows the structure under 50% extension from the top edge and 50% extension from the bottom edge, which. 100% uniaxial tensile strain in total.

FIG. 12D) in turn shows the structure under 100% extension from the top edge and 100% extension from the bottom edge, giving 200% uniaxial tensile strain in total.

In FIG. 13, an embodiment example showing a rotating rectangular auxetic structure with second-order fractal horseshoe-serpentine connections (FIG. 13A) and the rotating rectangular auxetic structure with second-order fractal horseshoe-serpentine connections in 3D cylindrical form (FIG. 13B) are shown.

In addition, FIG. 14 shows an embodiment example that includes a rotating rectangular auxetic structure with second-order fractal horseshoe-serpentine connections and island structure areas of different sizes (FIG. 14A), and the rotating rectangular auxetic structure with second-order fractal horseshoe-serpentine connections and island structure areas of different sizes formed into a 3D cylindrical geometry (FIG. 14B). In the center of the structure, the island structure areas are shown with a different size and shape than at the edge of the structure or even than in the previous figures.

FIG. 15 shows a possible embodiment of the rotating rectangular auxetic structure with second-order fractal horseshoe-serpentine connections from FIG. 13. The lowest edge is held as a fixed constraint, while a uniaxial force is applied to the top edge. FIG. 15A) shows the structure in the equilibrium state. FIG. 15B) shows the structure under 100% compression. FIG. 15C) shows the structure under 40% uniaxial stress and FIG. 15D) shows the structure under 75% uniaxial stress, where the structure exhibits auxetic behavior.

Examples of a 2D wavy mesh design with a square lattice geometry using second-order fractal horseshoe-serpentine connections and square island structure areas and a 2D wavy mesh design with a square lattice geometry using second-order fractal horseshoe-serpentine connections and square island structure areas formed into a 3D cylindrical geometry are shown in FIG. 16 (FIGS. 16A)-16B)).

In FIG. 17, the auxetic behavior of a structure in 2D wave lattice design with square lattice geometry using second-order fractal horseshoe-serpentine joints and square island structure faces as shown in FIG. 14 with enhanced extension and compression is presented. Uniaxial strain is applied to both the left and right edges of the structure in the direction indicated by the arrow. Showing:

17A) Structure under 30% compressive strain from the top edge and 30% compressive strain from the bottom edge. Uniaxial compressive strain of 60% in total.

17B) Structure under 20% compression load from the top edge and 20% compression load from the bottom edge. Uniaxial compressive strain of 40% in total.

17C) Structure under 10% strain from top edge and 10% strain from bottom edge. 20% total uniaxial tensile strain.

17D) Structure under 30% elongation from top edge and 30% elongation from bottom edge. Uniaxial tensile strain of 60% in total.

FIG. 18 demonstrates auxetic behavior with increased extensibility in a 2D wave lattice with square lattice geometry using second-order fractal horseshoe-serpentine connections and square island structure faces. The black inset shows the innermost unit cell of the lattice structure, while the gray inset shows the edge unit cell closest to the direction of the applied force of the lattice structure under strain. The top edge of the structure is held as a fixed constraint, while the bottom edges of the structure are uniaxially stretched in the direction indicated by the arrow. Non-uniform strain behavior and out-of-plane bending are observed.

18A) Structure in equilibrium state

18B) Structure under ˜5% strain.

18C) Structure at ˜10% extension

18D) Structure under ˜20% expansion

18E) Structure under ˜100% expansion

FIG. 19 represents a finite element simulation of auxetic behavior with increased compressibility in a 2D wave lattice with square lattice geometry using second order fractal horseshoe-serpentine connections and square island structure areas. A uniaxial compressive load is applied to the lowest edges of the web structure in the direction indicated by the arrow. Non-uniform strain behavior is observed. Showing:

19A) Structure at equilibrium (0% load).

19B) structure under ˜10% compression

19C) Structure under ˜20% compression

19D) Structure under ˜40% compression.

Thin films of auxetic shape memory alloys will be the preferred substrate for future medical implants. Shape memory alloys such as TiNi are currently used in passive medical devices such as stents because they are biocompatible and can be made small enough to be inserted into the body's arteries via a microcatheter. The main advantage of using shape memory alloys as a functionalized auxetic backbone for medical implants is that they add functionality to the implant (i.e., self-deployment, shape recovery, superelasticity). Large strains of 6-8% are achieved through thermal and stress-induced phase transformations, therefore auxetic SMAs can expand or deconvolve to much larger volumes than conventional auxetic metals (for example, steel). In addition, non-biocompatible NiTi-based shape memory alloys such as TiNiCu and TiNiCuCo can be engineered to exhibit extremely low fatigue behavior that allows them to undergo ˜10 million transition cycles with minimal change in mechanical or thermal behavior, making them attractive for wearable devices.

FIG. 20 illustrates the fabrication of a functionalized smart stent using an auxetic shape memory alloy substrate. FIG. 20a) shows that the starting material is an amorphous auxetic thin film SMA structure fabricated in a flat 2D state by a prior art fabrication method (e.g., photolithography or laser cutting). The typical reentrant auxetic geometry has been modified to have low-stress arrow-shaped island structure faces, which we call “reentrant arrow island structures.” FIG. 20b) shows that the amorphous sheet can be formed into a complex 3D shape by crystallization at high temperature (400° C.-800° C.), e.g., by rapid thermal annealing around a stainless steel object in the desired shape. FIG. 20c) shows that the crystallized auxetic 3D structure can be restored to a flat 2D state by applying mechanical force (i.e., clamping the component to a flat substrate). At this point, the substrate is ready for typical advanced micromachining for direct fabrication and integration of MEMS and NEMS components on the island structure areas of the auxetic structure.

Because the SMA backbone is conductive, both sides of the SMA substrate can be functionalized, increasing the available surface area for device integration. Since the SMA can be a common ground electrode, it can function as a “printed circuit board.” Examples of devices suitable for functionalization include integrated biosensors and readout components, antennas, piezoelectric devices, batteries, cameras, LEDs, actuators, and the like. FIG. 20d) shows that after fabrication of all functional devices, the mechanical clamp is removed so that the functionalized auxetic shape memory substrate can elastically recover its 3D shape when fabricated with a superelastic SMA. Since low stresses occur on the island structure areas during bending, stretching, twisting and folding motions, the integrated circuits patterned on the island structure areas of the SMA substrate also benefit from low stresses during deformation. If the final austenite temperature is above room temperature, the functionalized medical implant must be heated to fully recover its original shape. Since the phase transformation is reversible, the shape memory effect and superelastic effect can be used to compress the component to the required small volume of the microcatheter and then heat it to body temperature (˜37° C.) to bring it to its final position.

FIG. 21 shows a cross-sectional view of the sectional structure of an auxetic web structure or field structure according to the invention with connections 13 and open spaces and island structure areas 11, 12 as an implantable structure made of a biocompatible material.

This is an example of a series of fabrication steps that can be performed to fabricate and electrically isolate two different devices on adjacent island structure areas after mechanically clamping the auxetic SMA backbone flat, as described in FIG. 20c).

In step a), the auxetic base structure consisting of a crystallized shape memory alloy/SMA/shape memory alloy is first fabricated, forming island structure areas 11, 12 and interconnections 13, with the interconnections connecting the island structure areas. In this process or quasi-manufacturing diagram, only two island structure areas 11, 12 are shown as examples, which are then functionalized. Some island structure areas may be functionalized accordingly or may be functionalized further on.

In step b), a first electrical insulation layer 2 is applied to at least those areas of the structure in which electrical connections and/or electrical components/electronic components are to be applied. A complete coating of the structure can also be carried out.

In step c), a first electrode 3 is applied so that at least the necessary areas are covered with an appropriate conductive material. So at least the areas of the connections and those island structure areas that require electrical connection are covered with the first electrode 3.

By this embodiment, an electrical structure is already realized, since the basic structure, namely the metallic auxetic SMA structure (with the shape memory alloy) 11, 12, 13 serves as a common ground electrode and the now applied electrode 3 represents the further conductor. The SMA auxetic backbone is itself then the common ground electrode, so as to connect electrical components.

In step d), the assembly of the electronic components 41, 42 then takes place, which can be or are of different nature and in particular can be interconnected in combination to form a large system.

In step e), the last step in this example is then the coating of the overall structure with a biocompatible material. For example, this may be a biocompatible polymer that protects the human body from electronics. The area of the metallic auxetic SMA structure can be omitted here, as this is biocompatible.

At this point, it should be noted that sensible patterning does not necessarily require that all areas of the auxetic SMA structure 11, 12, 13 be provided with appropriate layers and/or components; rather, there may be empty island structure areas and interconnects that have a supportive function but do not have a load-bearing function for electrical components or even electrical conduction. This differs from the methods in US 2020/0144431 A1, where a semiconductor device had to be patterned with serpentine interconnects on each island structure area and each serpentine of an auxetic lattice structure.

Not further illustrated, but furthermore also possible, is the positioning of the first electrode above the electrical components or electronic components, so that an electrically insulating layer is first built up on the auxetic SMA structure and then the electronic components or, in general, the components are applied, followed by a further electrically insulating layer followed by the electrode. In addition, further conductors, each separated by electrically insulating layers, can be built up on top of each other. Deviations from this are of course possible for design reasons.

Upon completion of fabrication of all MEMS components, the SMA is heated above its martensitic phase transformation temperature (typically body temperature ˜37° C.) to restore the shape previously established during annealing. If the part is intended as a medical implant, it can now be crimped to be inserted into a microcatheter. If a flat 2D shape was thought of, the functionalized stretchable/flexible device is ready to conform to the underlying substrate (e.g., the skin of a wearable e-tattoo or biosensor). At this stage, the functionalized devices can optionally be embedded (or coated) in a biocompatible polymer if required for the application.

Various thin-film processes known in the prior art can be used to apply the various functional layers. Preferably, sputtering, in particular magnetron sputtering, should be mentioned in this context. However, other thin-film processes are also possible, such as sol-gel or vapor disposition processes. Reference should also be made to U.S. Pat. No. 8,758,636 B2, which discloses a method of fabricating a medical functional element having a self-supporting lattice structure, specifically describing the fabrication of special shape memory alloy thin film components intended for stent devices.

FIG. 22 shows the structure known from FIG. 21 section d), supplemented by at least one further electrode 6. This electrode 6 can additionally perform further functions of the electronic components, for example, communication or additional transmission of energy or the like. Further electrodes can also be provided, in which case they can connect different electronic components with each other.

Complex network structures can be constructed.

FIG. 23 shows a first exemplary embodiment of an implantable structure according to the invention in the form of a tube inserted in an artery using a modified reentrant auxetic geometry as already introduced in FIG. 20.

FIG. 23a) shows a reentrant auxetic arrow island structure in a 3D cylindrical geometry with low-stress island structure areas (in the form of arrows) functionalized with MEMS components according to the process flow presented in FIG. 20.

This embodiment can be used as a smart stent, with the 2D detail in FIG. 23b showing three different electronic components (represented by a square, a circle, and a triangle) attached to the arrow-shaped island structure areas of the modified low-stress auxetic structure. FIG. 23c) shows the component that is inserted into the artery. The auxetic backbone of the smart stent allows conformal contact with the nonlinear curved surface of the artery. Next-generation medical implants must be equipped with an array of electronic devices that measure vital patient data and transmit it to the physician in real time. For example, a functional cardiovascular stent could monitor a patient's blood pressure, as well as many other vital signs, from home and then wirelessly transmit the data to the hospital or a physician.

Auxetic shape memory alloys would be particularly attractive for use in medical implants and stents in arteries of the cardiovascular and neurological systems because the implants could be delivered to the patient through noninvasive endovascular treatments. Negative Poisson's ratio allows auxetic structures to change shape and conform to the surrounding surface, the complex arteries and veins of the body, as shown in FIG. 23c). After a stent has been placed in a patient's body for several years, the stent may trap large and small blood clots. Over time, this can lead to complete blockage of the arteries and cause serious and life-threatening medical complications such as pulmonary embolism, heart attack, or stroke. The accumulation of endogenous material (e.g., tissue, cells) on the stent could be monitored with a force sensor in a smart auxetic stent. An antenna could emit a signal to notify the physician when a critical threshold is reached. The functional stent could also be self-powered if a piezoelectric energy collector is integrated into the island or backbone of the auxetic structure.

In addition, it is possible to directly fabricate MEMS and NEMS components to provide the island structure areas of the auxetic structure with integrated sensors and readout components.

As another example, what is disclosed herein could provide a platform for integrating components such as cameras, lights (LEDs), and sensors to guide/position/track the stent position during placement. With further innovation of this invention, this could be a method that eliminates the need for MRI or X-ray imaging for stent placement in the future. In addition to conforming to the shape of the body, the auxetic structure can also be designed to achieve a compromise between the areal density of the unit cell and the maximum volume expansion of the entire structure. For example, changes in blood flow (pressure) in some arteries can be controlled by designing the areal density of the auxetic structure (i.e., large vs. small island structure areas or large vs. small openings between island structure areas) in the deployed state. Modifying the areal density of the device to restrict blood flow to an aneurysm is desirable, for example, in the case of a flow diverter stent. In addition, noninvasive measurements of medical data, such as blood pressure, could be wirelessly transmitted to the physician if the appropriate sensing elements are functionalized on the islet structure areas, which have only low voltages. In addition, these island structure areas can be equipped with X-ray markers that detect rotation of the stent or device so that the devices can be deployed in a specific position with a specific angle so that the functional islands can be easily placed where they are needed.

The large tensile stresses associated with the superelastic effect enable auxetic shape memory materials to provide extremely high strength, high working performance, and high actuation density. It has already been shown that auxetic stent structures have higher radial forces at the stent circumference 10 times higher than current stent designs. This means that thinner SMA devices can be fabricated that are easier to squeeze, maneuver, and deploy in the microcatheter.

The combination of flexibility and strength offered by shape memory alloy auxetic stents could also be useful for other medical applications that require temporary or permanent opening of a narrow passageway. These include stents that require greater radial forces than those required for the brain, for example, for use in the esophagus, urethra, and prostate.

Because of the above properties of auxetic shape memory materials, the present invention is suitable for functionalizing the exterior of surgical devices or for other temporary medical implants. For example, a flexible/stretchable 3D implant with integrated CO2/O2 sensors, cameras, LEDs, and pressure sensors could be a useful tool for surgeries/procedures where tubes are inserted into the patient's airway as described by Ullah, Ramzan, et al. in “Real-Time Optical Monitoring of Endotracheal Tube Displacement.” Biosensors 10.11 (2020): 174. For example, if such a device were wrapped around the outside of an intubation tube, it could help physicians/respiratory therapists insert the intubation tube more quickly and accurately. The cameras/LEDs help to find the right place in the trachea for placement (2 cm-5 cm above the carina). The CO2/O2 sensors integrated on the outside of the intubation tube (i.e., directly on (or above) the cuff) could help detect air leak (indicating misplacement of the device either in the lung or esophagus). The current method of detecting correct placement of the tube relies on x-rays. Built-in pressure sensors in the cuff could help physicians monitor the pressure they are applying to the patient as they inflate the cuff (the balloon) to secure the intubation tube. This is just one example of how an auxetic medical SMA implant could be used in conjunction with existing surgical instruments.

Like the stent placement issues mentioned earlier, confirming intubation tube placement often requires radiography, which is both time-consuming and costly. With the proper arrangement (co-integration) of multiple functional devices, the medical implant disclosed in this invention may eliminate this imaging step in the future.

Treatment of brain aneurysms also requires a miniaturized medical device that can be deployed noninvasively with current endovascular treatment methods, such as a catheter.

Flow detour stents, coiling, and woven endobridges (WEBs) are common methods of treating aneurysms. While most types of cerebral aneurysms can be treated with flow diverter stents, there are currently no suitable stent-based intrasaccular devices for the treatment of aneurysms that have the shape of a geodesic dome. Stents also cannot be used for bifurcation-type aneurysms because they can block blood flow in an artery, which can lead to stroke. The most common method of treating this type of aneurysm is to physically occupy the space (e.g., with Pt coils) to restrict further blood flow into the aneurysm. However, a major side effect of this treatment is that the Pt coils are a source of noise that prevents accurate measurement of blood flow in the aneurysm using conventional MRI techniques. The auxetic design according to the invention is capable of providing the same advantages as a WEB device as well as additional advantages because the auxetic design allows more freedom in sizing the device by photolithography than a conventional mesh design. In addition, the low-stress island structure areas of the auxetic structure of the invention are the only type of intrasaccular medical devices that are functionalized and allow direct readout of information related to the healing process, such as blood flow in the aneurysm, device failure, or reduction in the body's response, such as inflammation, to the device.

The disclosed invention is a unique type of device that can overcome the limitations presented for the modern treatment of intrasacular aneurysms. The modified stretchable auxetic structure according to the invention exhibits synclastic (conformal) bending around dome-shaped curves (as shown in the embodiment example in FIG. 7). This type of bending behavior is extremely difficult or impossible to achieve with current stent technologies. With the use of the structure of the invention, conformal bending about a dome shape required for intrasaccular devices is made possible.

The synclastic bending properties of auxetic SMA implants could also be useful for improving surgical instruments. Because the auxetic substrate can conform to the shape of its surroundings, it could wrap around the outside of existing surgical instruments. This could improve surgical outcomes by allowing physicians to add new devices to their surgical instruments that were not previously available (e.g., lights, cameras, sensors). By integrating sensors on the islet structural surfaces, it would be possible to monitor blood pressure or blood flow inside an aneurysm noninvasively with this device. Noninvasive monitoring of blood flow without MRI imaging could be revolutionary for the treatment of brain aneurysms. In addition, the use of a dome-shaped SMA device inside the aneurysm would be safer for the patient by reducing surgical time and increasing patient safety by reducing anesthesia time, radiation, and contrast exposure.

FIG. 24 shows a second embodiment example of an “S-shaped island structure plane” auxetic geometry, modified to provide island structure planes for device integration from conventional “S-shaped” auxetic structures, shown as a 2D plane (FIG. 24A), a cutaway (FIG. 24B), and a 3D smart stent (FIG. 24C). FIG. 22B also shows that three different types of functional components are integrated on the substrate. Conventional “S-shaped” auxetic structures are known from Meena, Kusum, and Sarat Singamneni. “A new auxetic structure with significantly reduced stress concentration effects”. Materials & Design 173 (2019): 107779.

FIG. 25 shows the results of finite element simulations of the design example of an “S-shaped-island-structure-surfaces” auxetic structure from FIG. 24 with the structure in equilibrium (FIG. 25A) and under uniaxial tension (y-direction) (FIG. 25B). It is shown that the modified auxetic structure retains its auxetic behavior.

FIG. 26 shows the “S-shaped island structure planes” auxetic geometry exhibiting synclastic bending when formed into a 3D sphere. This suggests a potential use of the structure as an intrasaccular aneurysm device.

Applications of the technology in particular of the substrate structure according to the present disclosure include:
functionalized medical devices and implants (conformal skin sensors, stents, percutaneous heart valve repair, intrasaccular devices, expandable/collapsible endotracheal tube, functional inferior vena cava (IVC) filter);
Space applications (deformable lightweight space applications, i.e. Substrate structure enabling more functionalization on rovers and spacecraft, deployable solar sails);
Energy collectors integrated directly into car and bicycle tires (e.g., piezoelectric elements);
flexible/stretchable LEDS, displays and cameras (individual pixels/sensors integrated on the Island structure areas integrated);
General structures for wearable and stretchable electronics;
Stretchable electrodes for soft actuators;
Functionalized transportation devices (e.g., indoor/outdoor aircraft, Spacecraft, boats, hot air balloons);
Functionalized outdoor equipment (e.g., solar-heated sleeping bag and tent);
Impact-resistant energy absorbers for vehicles;
Architectural buildings (structures can likely bend around any shape);
Auxetic clothing and functionalized garments (e.g., compression socks, -shirts, pants, shoes, backpacks, tourniquets);
Impact-resistant military equipment (e.g., bulletproof vest).

The structures disclosed herein can be used in the same applications that conventional auxetic structures are used. In particular, the applications are:
Biomedical: arterial dilator/stent, drug-eluting stent, stent-assisted coiling, smart wound dressing, tissue engineering (i.e. artificial skin), artificial blood vessels, wound pressure pads, surgical devices;
Aerospace: gas turbine engine blades, thermal protection, aircraft noses, wing fairings, acoustic vibration rivets;
Automotive: bumpers, cushions, thermal protection, vibration dampers;
Sensors/Actuators: Wave propagation, vibration/damping structures, smart strain sensors, hydrophone, piezoelectric components;
Military/defense/sports: impact/energy absorbing military equipment with integrated sensor components (e.g., bulletproof vest, helmet, knee pads, etc.).

If the auxetic web structure or field structure according to the invention is used for functionalized medical devices and implants, they must be able to withstand extreme demands. Biosensors, also called wearable devices, for example, that can be integrated directly on the skin or a substrate worn close to the skin, must be able to adapt to extreme movements and the resulting changes in the body's radius of curvature. Stretchable devices can be fabricated using serpentine structures, mesh structures, and island bridge structures. When fabricated from shape memory alloys, the auxetic structures shown here have many advantages over the stretchable designs for stretchable electronics known in the prior art. Herein, a platform for an extremely compliant and deformable substrate is demonstrated to enable the development of novel stretchable thin film devices. Auxetic shape memory alloy substrates offer superior mechanical properties over the state of the art for flexible/stretchable designs based on copper serpentines in island-bridge configurations. Shape memory alloys have superior mechanical properties over copper that allow them to recover elastically from extremely applied large amounts of strain (theoretically up to >8% elastic).

There are many applications of shape memory/piezoelectric composites for various energy harvester and microactuator applications. For example, sensitive magnetoelectric sensors based on piezoelectric and magnetostrictive materials can be integrated on shape memory alloys. Miniaturized magnetoelectric composites are appropriately required for applications such as bio-magnetic sensors, antennas, energy harvesters, and surface acoustic wave sensors. In principle, all auxetic sensor concepts can be energized by the motion of the body or blood flow itself when paired with a piezoelectric (or magnetoelectric) energy harvester. Selective fabrication of AlN components on the low voltage islands may enable the development of sensitive magnetoelectric sensors for biomagnetic sensing of sensitive signals coming from the heart and brain. Such magnetoelectric sensors would also be useful for applications such as deep brain stimulation.

The unique properties of shape memory alloy can increase the mechanical strength/robustness of a stretchable circuit while extending the life of the device/devices. Since SMAs have significantly larger intrinsic strains compared to traditional metals such as copper, tremendous global strains can be achieved through SMA archimedean spiral interconnects and/or self-similar fractal design interconnects. This enables structures with greater areal density into which more active circuit components can be integrated, enabling stretchable devices with higher efficiency. Thanks to a structure with higher areal density, more complex integrated circuits can also be realized. Through compositional design and proper heat treatment, nickel-titanium-based shape memory alloys, such as TiNiCu or TiNiCuCo, can be designed to undergo reversible phase transformation for up to 10 million cycles. Therefore, the implementation of shape memory alloy auxetic substrates would be extremely useful to enable new wearable and deformable electronic applications.

The auxetic structure improves the conformability of the electronic device to the skin or wearable substrate, preventing premature failure due to delamination problems. Better contact with the skin enables better functioning sensors that measure sensitive bio-magnetic signals. Appropriate wearable sensors for magnetoencephalography (MEG), magnetocardiography (MCG), electroencephalography (EEG), and electrocardiogram (ECG) MCG sensors are possible by fabricating active sensor components on the island structure areas of the auxetic structure. Ultrasonic sensors integrated into the structure would also allow the wearable device to see under the skin. Moreover, these types of designs are also united, useful and preferred for an electronic skin, soft electronics, exoskeletons and other soft robotics applications. Furthermore, if the application requires, it is possible to electrically isolate island structure areas from each other. Similarly, island structure planes could particularly preferentially share a common ground electrode through the SMA.

An active electronic component is not required on each of the auxetic island structure areas. Moreover, the electronic device need not be specifically a semiconductor device. For example, an implantable structure could be formed that includes a sensor on one island structure area, an actuator on another island structure area, and a transistor (or any other type of electronic device) on another island structure area. In addition, a significant advantage can also be seen in the fact that the low loading on each island structure area allows essentially any type of active or passive device, and they are electrically interconnected via multiple island structure areas, which in turn allows for a large area that can be used for appropriate components. In addition, many other types of materials and devices could now become “stretchable” (i.e., the material need not be a semiconductor, as is the case, for example, in US 2020/144 431 A1). Essentially, any type of component can remain in its original, traditional, rigid structure.

The reference signs used in the figures are provided below:
1 island structure area of a SMA-auxetic structure
11 first island structure area of a SMA-auxetic structure
12 second island structure area of an SMA-auxetic structure
13 interconnection of an SMA-auxetic structure
2 electrically insulating material
3 first electrode
41 first electronic component
42 second electronic component
43 third electronic component
5 biocompatible inclusion
6 supplementary electrode
7 substrate

1. An auxetic web structure or field structure comprising:
island structure areas and
interconnections between the individual island structure areas,
wherein
there are open spaces between the individual island structure areas, and
the connections with the island structure areas form a web structure or field structure;
wherein
the connections are extensible and are configured as Archimedean spiral connections and/or self-similar fractal design connections;
the connections intersect the island structure areas at the same location and at the same angle intersect the auxetic framework at the same location and angle as the previous rigid connectors;
the island structure areas are not bendable or are only slightly bendable when subjected to forces,
wherein the stresses on the island structure areas are at least one order of magnitude below those of the extensible connection
and/or
are not or only insignificantly variable in their magnitude
and
the Poisson's ratio of the structure under uniaxial deformation is negative.

2. An auxetic web structure or field structure according to claim 1, wherein the individual island structure areas and/or the individual connections are designed to vary in size and shape.

3. An auxetic web structure or field structure according to claim 1, wherein at least in sections over the auxetic web structure or field structure, areas are formed identically and/or periodically.

4. An auxetic web structure or field structure according to claim 1, wherein the self-similar fractal design connections comprise Koch's lines, Peano's lines, Hilbert's lines, Moore's loops, Vicsek's loops and branched meshes.

5. An auxetic web structure or field structure according to claim 1, wherein the auxetic web structure or field structure is planar, 2D fabricated and subsequently adapted to 3D surfaces.

6. An auxetic web structure or field structure according to claim 1, wherein at least individual island structure areas for receiving electronics or individual island structure areas equipped with electronics are provided.

7. An auxetic web structure or field structure according to claim 6, wherein the electronics located on the island structure areas are electrically connected to each other via at least one conductor track electrically insulated from the auxetic web structure or field structure on at least one connection of the auxetic web structure or field structure.

8. An implantable structure made of a biocompatible material having the auxetic web structure or field structure according to claim 1, wherein the biocompatible material is a metallic shape memory alloy and the auxetic web structure or field structure is metallic.

9. The implantable structure according to claim 8, wherein the implantable structure has a self-expanding implant shape with a cylindrical and/or spherical and/or hemispherical and/or tubular and/or curved tubular structure at least in sections.

10. The implantable structure according to claim 8, wherein the auxetic web structure or field structure is provided in the form of a common ground electrode.
